SEQUENCE LISTING (33 sequences)

SEQ ID NO: 1 - RNA, 20 nt, synthetic construct
  source 1..20: mol_type = other RNA
cgtagcccacggcgatgaag

SEQ ID NO: 2 - RNA, 20 nt, synthetic construct
  source 1..20: mol_type = other RNA
atttcttcacatccgtgtcc

SEQ ID NO: 3 - RNA, 20 nt, synthetic construct
  source 1..20: mol_type = other RNA
gtctcctggtcccaatactc

SEQ ID NO: 4 - RNA, 20 nt, synthetic construct
  source 1..20: mol_type = other RNA
gtagcccacggcgatgaagc

SEQ ID NO: 5 - RNA, 20 nt, synthetic construct
  source 1..20: mol_type = other RNA
tagcccacggcgatgaagcg

SEQ ID NO: 6 - RNA, 20 nt, synthetic construct
  source 1..20: mol_type = other RNA
gagggttcggggcgccatga

SEQ ID NO: 7 - RNA, 20 nt, synthetic construct
  source 1..20: mol_type = other RNA
ccggaacacacggaatgtga

SEQ ID NO: 8 - RNA, 20 nt, synthetic construct
  source 1..20: mol_type = other RNA
acagcgacgccgcgagccag

SEQ ID NO: 9 - RNA, 20 nt, synthetic construct
  source 1..20: mol_type = other RNA
ccactcacagattgaccgag

SEQ ID NO: 10 - RNA, 20 nt, synthetic construct
  source 1..20: mol_type = other RNA
cgacgccgcgagccagagga

SEQ ID NO: 11 - RNA, 20 nt, synthetic construct
  source 1..20: mol_type = other RNA
cagattgaccgagtggacct

SEQ ID NO: 12 - RNA, 20 nt, synthetic construct
  source 1..20: mol_type = other RNA
cagactgaccgagtggacct

SEQ ID NO: 13 - RNA, 20 nt, synthetic construct
  source 1..20: mol_type = other RNA
acagactgaccgagtggacc

SEQ ID NO: 14 - RNA, 20 nt, synthetic construct
  source 1..20: mol_type = other RNA
gccgggccgggacacggatg

SEQ ID NO: 15 - RNA, 20 nt, synthetic construct
  source 1..20: mol_type = other RNA
ccaggagacacggaatgtga

SEQ ID NO: 16 - RNA, 20 nt, synthetic construct
  source 1..20: mol_type = other RNA
ccagtcacagactgaccgag

SEQ ID NO: 17 - RNA, 20 nt, synthetic construct
  source 1..20: mol_type = other RNA
tcgacagcgacgccgcgagc

SEQ ID NO: 18 - RNA, 20 nt, synthetic construct
  source 1..20: mol_type = other RNA
gagatacatctacaaccggg

SEQ ID NO: 19 - RNA, 20 nt, synthetic construct
  source 1..20: mol_type = other RNA
tggagagatacatctacaac

SEQ ID NO: 20 - RNA, 20 nt, synthetic construct
  source 1..20: mol_type = other RNA
ggttgtagatgtatctctcc

SEQ ID NO: 21 - RNA, 20 nt, synthetic construct
  source 1..20: mol_type = other RNA
ggagagatacatctacaacc

SEQ ID NO: 22 - RNA, 20 nt, synthetic construct
  source 1..20: mol_type = other RNA
aggaatgctacgcgtttaat

SEQ ID NO: 23 - RNA, 20 nt, synthetic construct
  source 1..20: mol_type = other RNA
cagatacatctataaccgag

SEQ ID NO: 24 - RNA, 20 nt, synthetic construct
  source 1..20: mol_type = other RNA
cgagtactggaacagccaga

SEQ ID NO: 25 - RNA, 20 nt, synthetic construct
  source 1..20: mol_type = other RNA
cccgttggtgaagtagcaca

SEQ ID NO: 26 - RNA, 20 nt, synthetic construct
  source 1..20: mol_type = other RNA
gtgctacttcaccaacggga

SEQ ID NO: 27 - RNA, 20 nt, synthetic construct
  source 1..20: mol_type = other RNA
aggtcgtgcggagctccaac

SEQ ID NO: 28 - RNA, 20 nt, synthetic construct
  source 1..20: mol_type = other RNA
aggtgacagttgctacccga

SEQ ID NO: 29 - RNA, 20 nt, synthetic construct
  source 1..20: mol_type = other RNA
tcccgttggtgaagtagcac

SEQ ID NO: 30 - RNA, 20 nt, synthetic construct
  source 1..20: mol_type = other RNA
aactacgaggtggcgtaccg

SEQ ID NO: 31 - RNA, 20 nt, synthetic construct
  source 1..20: mol_type = other RNA
ctgttccagtactcggcaac

SEQ ID NO: 32 - RNA, 20 nt, synthetic construct
  source 1..20: mol_type = other RNA
actacgaggtggcgtaccgc

SEQ ID NO: 33 - RNA, 20 nt, synthetic construct
  source 1..20: mol_type = other RNA
cgaagcgcacgtactcctct

(19) United States
(12) Patent Application Publication
(10) Pub. No.: US 2026/0097074 A1
(43) Pub. Date: Apr. 9, 2026

(54) ERYTHROID LINEAGES DERIVED FROM PLURIPOTENT CELLS

(71) Applicant: Stratus Therapeutics, Inc., Cambridge, MA (US)

(72) Inventor: Dhvanit Shah, Chestnut Hill, MA (US)

(21) Appl. No.: 19/117,852

(22) PCT Filed: Oct. 5, 2023
(86) PCT No.: PCT/US23/76093

§ 371 Date: Apr. 2, 2025

Related U.S. Application Data
(60) Provisional application No. 63/413,439, filed on Oct. 5, 2022.

(51) Int. Cl. A61K 35/14; C12N 5/0789; C12N 15/86

(52) U.S. Cl. CPC A61K 35/14; C12N 5/0647; C12N 15/86; C12N 2500/24; C12N 2500/32; C12N 2501/105; C12N 2501/115; C12N 2501/125; C12N 2501/14; C12N 2501/155; C12N 2501/165; C12N 2501/2306; C12N 2501/2311; C12N 2506/45; C12N 2710/10043

(57) ABSTRACT

The present disclosure, in various aspects and embodiments, provides methods for generating hematopoietic lineages for cell therapy, including erythroid progenitor cells, progenitor erythroblasts, granulocyte-macrophage progenitor cells (GMPs), and megakaryocyte erythroid progenitor cells (MEPs), and erythroid cells. In various embodiments, the invention provides for efficient ex vivo processes for developing such hematopoietic lineages, including but not limited to progenitor erythroblast cells and erythroblast cell lineages, from human induced pluripotent stem cells (iPSCs). Cells generated according to the disclosure in various embodiments are functional and/or more closely resemble the corresponding lineage isolated from peripheral blood or bone marrow. The present invention also provides isolated cells and cell compositions produced by the methods disclosed herein, as well as methods for cell therapy.

### CROSS-REFERENCE TO RELATED APPLICATIONS

This application claims the benefit of and priority to U.S. Provisional Patent Application No. 63/413,439 filed Oct. 5, 2022, the contents of which are hereby incorporated by reference in their entirety.

### SEQUENCE LISTING

The instant application contains a Sequence Listing which has been submitted in XML format via EFS-Web and is hereby incorporated by reference in its entirety. Said XML copy, created on Sep. 26, 2023, is named GRU-012PC_Sequence_Listing.xml and is 30,040 bytes in size.

### BACKGROUND

According to the American Red Cross it is facing a national blood crisis—worst blood shortage in over a decade, posing a concerning risk to patient care. Amid this crisis, doctors are forced to make difficult decisions about who receives blood transfusions and who will need to wait until more products become available. Thus, developing off the shelf red blood cells (RBCs) or erythrocytes or progenitors thereof is an urgent need. Hematopoietic stem cells derived from induced pluripotent cells provides with such opportunity. However, current methodologies for the generation RBCs from iPSCs have proven inadequate because of constraints in expansion of erythrocyte lineage cells or insufficient enucleation. Accordingly, successful generation of red blood cell products from iPSCs ex vivo would fill a great need.

### SUMMARY OF THE DISCLOSURE

The present disclosure, in various aspects and embodiments, provides methods for generating hematopoietic lineages for cell therapy, including gene edited hematopoietic stem cells (HSCs), erythroid progenitor cells, progenitor erythroblasts, granulocyte-macrophage progenitor cells (GMPs), megakaryocyte erythroid progenitor cells (MEPs), and erythroid cells. In various embodiments, the invention provides for efficient ex vivo processes for developing such hematopoietic lineages, including but not limited to progenitor erythroblast cells and erythroblast cell lineages, from human induced pluripotent stem cells (iPSCs). Cells generated according to the disclosure in various embodiments are functional and/or more closely resemble the corresponding lineage isolated from peripheral blood or bone marrow. The present invention also provides isolated cells and cell compositions produced by the methods disclosed herein, as well as methods for cell therapy.

In other aspects and embodiments, this disclosure provides HSCs or erythroid progenitors that are derived from iPSCs that are gene edited to encode a hyperresponsive EPO Receptor. These HSC or erythroid progenitor cell populations can be used for more efficient ex vivo red cell production, or in other aspects, can be used to deliver HSCs or erythroid progenitors to patients in need to reduce or eliminate requirements for periodic blood transfusions.

In one aspect, the disclosure provides a method for preparing a cell population of a hematopoietic lineage. The method comprises preparing a pluripotent stem cell (PSC) population, such as an induced pluripotent stem cell (iPSC) population differentiated to embryoid bodies (EBs), and enriching for CD34+ cells to thereby prepare a CD34+ enriched population. Endothelial-to-hematopoietic transition (EHT) is induced in the CD34+ enriched population to thereby prepare a hematopoietic stem cell (HSC) population, optionally followed by a further enrichment of CD34+ cells. The resulting HSC population (or fraction thereof) in some embodiments can be differentiated to an erythroid hematopoietic lineage (e.g., erythroid progenitors) in some embodiments. In various embodiments, the hematopoietic lineage is selected from erythrocytes (i.e., red blood cells), progenitor erythroblasts, granulocyte-macrophage progenitor cells (GMPs), and megakaryocyte erythroid progenitor cells (MEPs).

In various embodiments, HSCs and erythroid lineage cells therefrom are derived from iPSCs which are gene edited to be one of: (i) HLA-A−B+C+DP−DR+DQ+, (ii) HLA-A−B+C+DP+DR+DQ−, (iii) HLA-A−B+C+DP−DR+DQ−; (iv) HLA-A−B−C+DP−DR+DQ+; (v) HLA-A−B−C+DP+DR+DQ−, (vi) HLA-A−B−C+DP−DR+DQ−. In some embodiments, HSCs and erythroid lineage cells are derived from iPSCs that are gene edited to be HLA-A^{neg}, homozygous for both HLA-B and HLA-C, and HLA-DPB1^{neg} and HLA-DQB1^{neg}. In some embodiments, the iPSCs are further homozygous for HLA-DRB1.

In some embodiments, the iPSCs of the present disclosure are gene edited to encode a hyperresponsive EPO Receptor. Erythropoiesis is the process for production of red blood cells. Erythropoietin (EPO) is the key hormone responsible for effective erythropoiesis. Erythropoietin receptor (EPOR) is a protein that in humans is encoded by the EPOR gene. The most well-established function of EPOR is to promote proliferation and rescue of erythroid (red blood cell) progenitors from apoptosis. Beneficial mutations in the EPOR increase red blood cell number and allow for improved oxygen delivery. For example, truncating mutations removing only parts of the intracellular EPOR C-terminus that bind negative regulators can render EPOR hyper-responsive to EPO.

These HSC populations or erythroid progenitors can be used for more efficient ex vivo red cell production, or in other embodiments, can be used to deliver HSCs or erythroid progenitors to patients in need to reduce or eliminate requirements for periodic blood transfusions. Further, because such cells can be gene edited to delete certain HLA genes (as described), the HSC populations and erythroid progenitors can be easily HLA-matched for a recipient. HSCs according to the disclosure differentiate to various hematopoietic lineages (similar to bone marrow CD34+ cells), and are capable of restoring hematopoietic system in a recipient.

In some embodiments, iPSC differentiation proceeds until cells are at least about 10% CD34+, or at least about 20% CD34+, or at least about 25% CD34+, or at least about 30% CD34+. In some embodiments, CD34 enrichment and EHT may be induced at Day 7 to Day 14 of iPSC differentiation, such as for example, Day 8, Day 9, Day 10, Day 11, Day 12, Day 13, or Day 14. Induction of EHT in CD34+ cells harvested from differentiated iPSCs, can be with any known process. In some embodiments, EHT generates HSCs through endothelial or hemogenic endothelial cell (HEC) precursors using mechanical, biochemical, pharmacological and/or genetic means (e.g., via stimulation, inhibition, and/or genetic modifications).

In some embodiments, the method comprises increasing the expression or activity of DNA (cytosine-5-)-methyltransferase 3 beta (Dnmt3b) in PSCs, embryoid bodies, CD34+-enriched cells, ECs, HECs or HSCs, which can be by mechanical, genetic, biochemical, or pharmacological means. In some embodiments, the induction of EHT comprises increasing the expression or activity of dnmt3b in hemogenic endothelial cells. In some embodiments, cells are contacted with an effective amount of an agonist of a mechanosensitive receptor or a mechanosensitive channel that increases the activity or expression of Dnmt3b. In some embodiments, the mechanosensitive receptor is Piezol.

In various embodiments, CD34+ cells (e.g., the floater and/or adherent cells) are harvested from the culture undergoing endothelial-to-hematopoietic transition and are optionally expanded in culture. Hematopoietic stem cells (HSCs) which give rise to erythroid, myeloid, and lymphoid lineages, can be identified and optionally sorted or enriched based on the expression of CD34+ cells and the absence of lineage specific markers (termed Lin−).

In some embodiments, the HSC population or fraction thereof is differentiated to red cells or progenitors or derivatives thereof. For example, the HSC population (or cells isolated therefrom) is cultured with EPO, IL-3 and SCF, for example, (and/or other extracellular matrix component(s)), and/or combinations thereof, to produce a population comprising erythroid progenitor cells or a derivative cell population (e.g., erythrocytes). The HSC population of cells give rise to high percentage of burst forming unit-erythroid (BFU-E) cells, and colony forming units-erythroid (CFU-E) cells, which are indications of induction of erythropoiesis.

In other aspects, the invention provides an erythroid lineage population, or pharmaceutically acceptable composition thereof, produced by the method described herein. In various embodiments, the composition comprises the desired cell population (e.g., erythrocytes) and a pharmaceutically acceptable vehicle. The pharmaceutical composition may comprise at least about 10⁷ erythrocytes per mL. The pharmaceutical composition may be provided in units of about 50 mL to about 500 mL, or from about 100 mL to about 500 mL, or from about 250 to about 500 mL.

In some aspects, an HSC composition or erythroid progenitor is provided with a hyperresponsive EPOR, and which may be produced by the method described herein. In some embodiments, the HSCs or erythroid progenitors are HLA-A^{neg}, homozygous for both HLA-B and HLA-C, HLA-DPB1^{neg}, and HLA-DQB1^{neg}. In some embodiments, the iPSCs are further homozygous for HLA-DRB1. The cell composition of this disclosure may further comprise a pharmaceutically acceptable carrier or vehicle suitable for intravenous infusion or other administration route, and the composition may include a suitable cryoprotectant. An exemplary carrier is DMSO (e.g., about 10% DMSO). Cell compositions may be provided in unit vials or bags and stored frozen until use.

Cells produced according to this disclosure can be administered or used in therapy, for example, for an inherited or acquired red cell disorder, bone marrow failure disorder, high-altitude-related physiological and pathological condition, anemia (e.g., sickle cell anemia), red cell enzyme deficiencies (e.g. G6PD), red cell membrane disorders (e.g. hereditary spherocytosis), hemoglobinopathies (e.g. sickle cell disease and thalassemia), hemolytic anemia, nutritional anemias (e.g. iron deficiency anemia, and folate deficiency), disorders of heme production (e.g. sideroblastic anemia), hemochromatosis, conditions related to chemicals or radiation exposure, and/or for treatment of subjects undergoing HSC transplant. In further embodiments, the red cells prepared according to this disclosure are provided as a pharmaceutical acceptable composition delivering or encapsulating drugs (including but not limited to enzymes), oxygen carriers, or other suitable materials to treat human disease or physiological or pathological conditions.

In certain embodiments, the present disclosure provides compositions and methods for treating anemia. In embodiments, the present disclosure provides for hematopoietic stem cell (HSC) compositions or erythroid progenitor compositions that provide for a durable and potent cell therapy for anemia. In embodiments, the HSCs are supercharged with an EPOR that is truncated and/or mutated, resulting in a hyperresponsive EPOR. In embodiments, the compositions and methods of the present disclosure can provide patients with sufficient red blood cells to supply healthy oxygenation levels.

In various embodiments, the present disclosure provides a method of treating a subject in need of red blood cell production, comprising administering to the subject the HSC or erythroid progenitor composition of the present disclosure. As such, the HSCs produced according to this disclosure may be used to produce red blood cells in vivo in a durable and potent manner. In some embodiments, the recipient subject has anemia. In some embodiments, the recipient subject has sickle cell anemia, aplastic anemia, anemia associated with a bone marrow disease or bone marrow failure, anemia from blood loss, or hemolytic anemia. In some embodiments, the recipient subject has Fanconi anemia. In some embodiments, the subject has thalassemia. In some embodiments, the HSCs or erythroid progenitors are used to prepare blood products for treating co-morbidities associated with blood, bone marrow, immune, metabolic, or mitochondrial disorders.

Other aspects and embodiments of this disclosure will be apparent from the following detailed disclosure and working examples.

### BRIEF DESCRIPTION OF DRAWINGS

FIG. 1 shows that ETV2 over-expression (OE) does not affect pluripotency. FIG. 1 shows FACS plots representative of transduction efficiency of iPSC with an adenoviral vector to overexpress ETV2 and GFP sequences. ETV2 overexpression does not affect the iPSC stemness as shown by the expression of the TRA-1-60 stemness marker.

FIG. 2 shows that ETV2 over-expression (OE) increases the yield of hemogenic endothelial cells. Representative flow cytometric analysis of hemogenic endothelial cells (described as CD235a−CD34+CD31+) and relative quantification demonstrates that ETV2-OE enhances the formation of hemogenic endothelial cells.

FIG. 3 shows that ETV2 over-expression (OE) enhances CD34+ cell formation during iPSC differentiation. Representative flow cytometric analysis of CD34+ cells and relative quantification demonstrates that ETV2-OE enhances the CD34+ cell formation.

FIG. 4A and FIG. 4B show that iPSC-derived HSCs that are derived with EHT of CD34+ cells (in this example with Piezol activation) undergo pro-T cell differentiation similar to bone marrow (BM)-HSCs. FIG. 4A is a FACS plot of differentiation efficiency to CD34+CD7+ pro T cells of Bone Marrow (BM) HSCs and iPSC-HSCs derived with EHT of 34+ cells (in this example with Piezol activation). FIG. 4B is a quantification of CD34+CD7+ cells (%) derived with (1) BM-HSCs and (2) iPSC-HSCs (Piezol Activation). FIG. 4B shows the average of three experiments.

FIG. 5A and FIG. 5B show that iPSC-derived HSCs generated with EHT (in this example with Piezol activation) undergo T cell differentiation and can be activated with CD3/CD28 beads similar to BM-HSCs. FIG. 5A is a FACS plot of activation efficiency (CD3+CD69+ expression) of T cells differentiated from BM-HSCs and iPSC-derived HSCs (in this example generated with Piezol activation). FIG. 5B is a quantification of CD3+CD69+ cells (%) derived with (1) BM-HSCs and (2) iPSC-HSCs (Piezol Activation). FIG. 5B shows the average of three experiments.

FIG. 6 shows that iPSC-derived HSCs generated with EHT of CD34+ cells (in this example with Piezol activation) can differentiate to functional T cells. IFNγ expression is a consequence of T cell activation after T cell receptor (TCR) stimulation via CD3/CD28 beads. IFNγ expression in T cells differentiated from iPSC-derived HSCs (EHT of D8 34+ cells, including with Piezol activation) enhances HSC ability to further differentiate to functional hematopoietic lineages (in this example T cells). FIG. 6 shows the average of three experiments.

FIG. 7A and FIG. 7B show the phenotype analysis of HLA edited (e.g., triple knockout) cells performed by FACS and immunofluorescence. FIG. 7A shows the overall expression of HLA class-I molecules (HLA-A, HLA-B, and HLA-C) on the cell surface, where the HLA edited cells are positive for overall HLA class-I expression to a similar degree as wild-type cells (gHSCs). FIG. 7B shows cell expression of HLA-A via immunofluorescence, where HLA-A is not expressed in the HLA edited clone.

FIG. 8 shows that the HLA edited clones preserve their pluripotency (maintain trilineage differentiation), as illustrated by immunofluorescence, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

FIG. 9 shows the immune compatibility of the HLA edited HSCs. HLA edited HSCs and control HSCs (WT, B2M KO, and HLA Class II null) were co-cultured with peripheral blood mononuclear cells (PBMCs) matching HLA-B and HLA-C, but with mismatched HLA-A, and PBMC-mediated cytotoxicity was measured by an annexin V staining assay.

FIG. 10 shows in vivo engrafting potential of HLA edited HSCs. Equal proportions of mCherry HLA edited HSCs and wild-type HSCs (gHSCs) were mixed for a competitive transplant into mice, where bone marrow (BM) and peripheral blood samples were evaluated by FACS to compare the relative amounts of each cell type present in the samples.

FIGS. 11A and 11B show that deletion of HLA-A does not impact Class I peptide presentation. FIG. 11A shows a schematic representation of immunopeptidome analysis.

FIG. 11B shows results of the immunopeptidome analysis, which reveals that little difference exists in the numbers of peptides and representative proteins presented by class I molecules of WT and HLA-edited cells.

FIGS. 12A and 12B show that deletion of HLA-DP and DQ does not impact Class II peptide presentation. FIG. 12A shows immunopeptidome analysis scheme. FIG. 12B shows that despite the deletion of HLA-DP and DQ, the cells preserve their ability to present a broad spectrum of peptide through HLA Class II.

FIG. 13 is a schematic representation of in vivo testing of antigen-mediated immune response: Delayed Type Hypersensitivity Assay (DTH), sensitizing stage and elimination stage respectively.

FIGS. 14A and 14B show that HLA-edited HSCs reconstitute a functional immune system as demonstrated by DTH reaction in immune deficient mice. FIG. 14A shows a delayed-type hypersensitivity assay on transplanted mice were performed, which is an assay that involves the cross-talk of different types of immune cells. Mice were sensitized by subcutaneous injection of sheep Red blood cells (antigen). A functional immune system results in the swelling of the left paw that was measured with a micro caliper. As can be seen in FIG. 14A, the non-transplant mice did not show any left paw swelling as they are immunodeficient. Conversely, the mice transplanted with Cord Blood CD34+ cells show tissue swelling and doubled the diameter of their left paw. FIG. 14B is a graphical evaluation of the results shown in FIG. 14A.

FIG. 15 shows the HSC differentiation potential into T cell subtypes. After a 35-day differentiation period pro-T cells were evaluated by cell sorting for the presence of CD4+, CD8+, and AB+ T cell populations. FIG. 15 compares the differentiation potential of bone marrow-derived CD34+ cells, embryoid body CD34+ cells, and HSCs prepared according to the present disclosure (e.g., using Piezol activation) (“gHSCs”).

FIG. 16 shows the degree of T-cell mediated cytotoxicity measured from a co-culture of HSC-derived T cells with CD19+ lymphoma cells in the presence of an anti-CD3/CD-19 bispecific antibody. T cells prepared from HSCs according to the present disclosure (“gHSC”) demonstrate a high level of cytotoxicity against the target cells.

FIG. 17 shows the ability of the HSCs to develop into pro-T cells as measured by their CD34-CD7+ markers.

FIGS. 18A and 18B demonstrates increased expression of T cell-specific transcription factors and Thymus engrafting molecules with the pro-T cells derived from HSCs according to the instant disclosure. FIG. 18A shows TCF7 mRNA expression and FIG. 18B shows CCR7 mRNA expression.

FIGS. 19A and 19B shows that HSC-derived Pro-T Cells engraft and differentiate in thymus. FIG. 19A illustrates the engraftment and analysis procedure. FIG. 19B shows FACS analysis of CD3 cell population of cells gated on CD45+ cell population, which shows the superior engraftment and differentiation potential of the HSC-derived Pro-T Cells in the thymus.

FIG. 20 shows HSC-derived T cells can be activated in vitro. Top panel shows FACS analysis of activated T cells from different sources, including from HSCs prepared according to the present disclosure. T cells of the present disclosure demonstrate comparable or superior activation as measured by increased CD107 expression. The lower panel shows Dynabeads activation, where activated T cells express inflammatory cytokines. HSC-derived T cells express higher levels of inflammatory cytokines as exemplified by TNF-alpha and interferon gamma expression levels.

FIGS. 21A and 21B show that WT and HLA-edited HSCs can differentiate to the monocyte/macrophage lineage, which also preserves the overall expression of both class I and class II molecules as identified by CD11b+CD14+ markers (FIG. 21A). FIG. 21B shows analysis of HLA-I and HLA-II on cells gated on CD11b+CD14+.

FIGS. 22A to 22C show that HLA-DQB1 and HLA-DPB1 deletion does not affect the expression of other HLA Class II molecules. FIG. 22A is a schematic showing differentiation of HLA-edited iPSCs to macrophages. FIG. 22B is an immunofluorescence experiment confirming the specific deletion of the DPB1 and DQB1 molecules. FIG. 22C shows that the same cells preserve the class II DRB1 expression.

FIG. 23 shows that HLA-edited HSCs can differentiate into megakaryocytes (MK) which can further differentiate into platelets. Images at the left show increased proportion of platelets in HSCs by light microscopy at 1000× magnification. The graph at the right shows a statistically significant increase in the proportion of platelets differentiated from HLA-edited HSCs compared to BM CD34+ and iPSC-34+ cell populations.

The term “gHSC” is used herein to refer to the iPSC-derived hematopoietic stem cells of the present disclosure.

The terms “wild type” (WT), “unedited”, “non-HLA-edited” are used interchangeability herein to refer to the non-gene edited cells of the present disclosure.

EB34+ cells refer to Embryonic body derived CD34+ cells. These comprise hemogenic endothelial cells.

### DESCRIPTION OF THE INVENTION

The present disclosure, in various aspects and embodiments, provides methods for generating hematopoietic lineages for cell therapy, including gene edited hematopoietic stem cells (HSCs), erythroid progenitor cells, progenitor erythroblasts, granulocyte-macrophage progenitor cells (GMPs), megakaryocyte erythroid progenitor cells (MEPs), and erythroid cells. In various embodiments, the invention provides for efficient ex vivo processes for developing such hematopoietic lineages, including but not limited to progenitor erythroblast cells and erythroblast cell lineages, from human induced pluripotent stem cells (iPSCs). Cells generated according to the disclosure in various embodiments are functional and/or more closely resemble the corresponding lineage isolated from peripheral blood or bone marrow. The present invention also provides isolated cells and cell compositions produced by the methods disclosed herein, as well as methods for cell therapy.

In other aspects and embodiments, this disclosure provides HSCs that are derived from iPSCs that are gene edited to encode a hyperresponsive EPO Receptor. These HSC populations can be used for more efficient ex vivo red cell production, or in other aspects, can be used to deliver HSCs or erythroid progenitors to patients in need to reduce or eliminate requirements for periodic blood transfusions.

In accordance with aspects and embodiments of this disclosure, the ability of human induced pluripotent stem cells (hiPSCs) to produce essentially limitless pluripotent stem cells (PSCs) is leveraged to generate boundless supply of hematopoietic cells, including but not limited to therapeutic human erythrocytes or red blood cells (“RBCs”) or their erythroid progenitors. Use of RBCs for therapeutic purposes has been severely limited by their restricted availability, cell numbers, and limited expansion potential. Moreover, compared to primary cells, hiPSCs can more readily undergo genetic modifications in vitro, thereby offering opportunities to improve cell-target specificity, cell numbers, as well as bypassing HLA-matching issues for example. Additionally, fully engineered hiPSC clones, as compared to primary cells, can serve as a stable and safe source (Nianias and Themeli, 2019). Further, because hiPSCs, unlike human Embryonic Stem Cells (hESCs), are of non-embryonic origin, they are also free of ethical concerns. Accordingly, use of hiPSCs according to this disclosure confers several advantages over primary cells to generate therapeutic hematopoietic lineages, such as erythroid lineages.

In one aspect, the disclosure provides a method for preparing a cell population of a hematopoietic lineage. The method comprises preparing a pluripotent stem cell (PSC) population, such as an induced pluripotent stem cell (iPSC) population differentiated to embryoid bodies, and enriching for CD34+ cells to thereby prepare a CD34+ enriched population. Endothelial-to-hematopoietic transition (EHT) is induced in the CD34+ enriched population to thereby prepare a hematopoietic stem cell (HSC) population, optionally followed by a further enrichment of CD34+ cells. The resulting HSC population (or fraction thereof) in some embodiments can be differentiated to an erythroid hematopoietic lineage. In various embodiments, the hematopoietic lineage is selected from erythrocytes (i.e., red blood cells), progenitor erythroblasts, granulocyte-macrophage progenitor cells (GMPs), and megakaryocyte erythroid progenitor cells (MEPs).

Conventionally, hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with aspects and embodiments of this disclosure, it is discovered that inducing endothelial-to-hematopoietic transition (EHT) of a CD34+ cell population, and which can be derived from iPSCs-embryoid bodies, can be used for the ex vivo generation of superior hematopoietic stem cells, and hematopoietic lineages including erythroid lineages.

In some embodiments, the disclosure provides a method for generating an erythroblast population (e.g., early erythroid progenitors, late erythroid progenitors, and morphologically recognizable erythroid precursors), or a derivative of this population. For example, the method comprises generating a hematopoietic stem cell (HSC) population comprising human long-term hematopoietic stem cells (LT-HSCs) from iPSCs (e.g., hiPSCs). The HSC population is derived by induction of endothelial-to-hematopoietic transition of CD34+ cells (e.g., CD34+ cells derived from embryoid bodies). The HSC population (or cells isolated therefrom) is cultured with EPO, IL-3 and SCF, for example, (and/or other extracellular matrix component(s)), and/or combinations thereof, to produce a population comprising erythroid progenitor cells or a derivative cell population (e.g., erythrocytes). The HSC population of cells gives rise to high percentage of burst forming unit-erythroid (BFU-E) cells, and colony forming units-erythroid (CFU-E) cells, which are indications of induction of erythropoiesis.

Aspects and embodiments of this disclosure may be exploited to generate reticulocytes and/or enucleated mature erythrocytes from cells differentiated or derived from the hematopoietic stem cell (HSC) population comprising human long-term hematopoietic stem cells (LT-HSCs) derived from iPSCs (e.g., hiPSCs). For example, iPSC-derived HSCs can be cultured under erythroid differentiation conditions in a bioreactor (e.g., stirred bioreactor), which would yield enucleated cells of about 80% purity when cultured under optimal physical conditions: pH ranging between about 7.0 to about 7.5, about 25% to about 75% oxygen (e.g., about 50% oxygen), without gas-sparging and with mechanical agitation (impeller speed approximately 300-450 rev/min). Supplementary compounds such as cytokines and growth factors may be added to the erythroid differentiation conditions to facilitate the yield of enucleated cells.

In various embodiments, the iPSCs are prepared by reprogramming somatic cells. The term “induced pluripotent stem cell” or “iPSC” refers to cells derived from somatic cells, such as skin or blood cells that have been reprogrammed back into an embryonic-like pluripotent state. In some embodiments, iPSCs are generated from somatic cells such as (but not limited to) fibroblasts or PBMCs (or cells isolated therefrom). In some embodiments, the iPSCs are derived from lymphocytes (e.g., T-cells, B-cells, NK-cells, etc.), cord blood cells, PBMCs, CD34+ cells, or other human primary tissues. In some embodiments, iPSCs are derived from CD34+ cells isolated from peripheral blood, bone marrow, or cord blood. In various embodiments, the iPSCs are autologous or allogenic (e.g., HLA-matched at one or more loci) with respect to a recipient (a subject in need of treatment as described herein). In various embodiments, the iPSCs can be gene edited to assist in HLA matching (such as deletion of one or more HLA Class I and/or Class II alleles or their master regulators, including but not limited beta-2-microglobulin (B2M), CIITA, etc.), or gene edited to delete or express other functionalities. For example, iPSCs can be gene edited to delete one or more of HLA-A, HLA-B, and HLA-C, and to delete one or more of HLA-DP, HLA-DQ, and HLA-DR. In certain embodiments, the iPSCs retain expression of at least one HLA Class I and at least one HLA Class II complex. In certain embodiments, iPSCs are homozygous for at least one retained Class I and Class II loci. In some embodiments, iPSCs are derived from cord blood CD34+ cells or CD36+ erythroblasts. iPSCs can be derived from CD34+ cells that form universal donor red cells (i.e., Type O−). Other blood types can also be used.

In various embodiments, HSCs and erythroid lineage cells therefrom are derived from iPSCs which are gene edited to be one of: (i) HLA-A−B+C+DP−DR+DQ+, (ii) HLA-A−B+C+DP+DR+DQ−, (iii) HLA-A−B+C+DP−DR+DQ−; (iv) HLA-A−B−C+DP−DR+DQ+; (v) HLA-A-B−C+DP+DR+DQ−, (vi) HLA-A−B−C+DP−DR+DQ−. For retained HLA (for example HLA-B, HLA-C, and HLA-DR), cells can be homozygous or retain only a single copy of the gene. For example, the modified cells are identified at least as (a) HLA-C+ and HLA-DR+, and optionally identified as one or more of (b) HLA-B−, (c) HLA-DP−, and (d) HLA-DQ−. In exemplary embodiments, the modified cells are HLA-B+, HLA-DP−, and HLA-DQ−.

In some embodiments, HSCs and erythroid lineage cells derived from iPSCs are gene edited to be HLA-A^{neg}, homozygous for both HLA-B and HLA-C, and HLA-DPB1^{neg} and HLA-DQB1^{neg}. In some embodiments, the iPSCs are further homozygous for HLA-DRB1.

As used herein, the term “neg,” (−), or “negative,” with respect to a particular HLA Class I or Class II molecule indicates that both copies of the gene have been disrupted in the cell line or population, and thus the cell line or population does not display significant functional expression of the gene. Such cells can be generated by full or partial gene deletions or disruptions, or alternatively with other technologies such as siRNA. As used herein, the term “delete” in the context of a genetic modification of a target gene (i.e., gene edit) refers to abrogation of functional expression of the corresponding gene product (i.e., the corresponding polypeptide). Such gene edits include full or partial gene deletions or disruptions of the coding sequence, or deletions of critical cis-acting expression control sequences.

In some embodiments, the iPSCs are gene edited using gRNAs that are 16, 17, 18, 19, 20, 21, 22, 23, 24, 25, 26 or more nucleotides in length. In some embodiments, the gRNAs comprise a modification at or near the 5′ end (e.g., within 1-10, 1-5, or 1-2 nucleotides of the 5′ end) and/or a modification at or near the 3′ end (e.g., within 1-10, 1-5, or 1-2 nucleotides of the 3′ end). In some embodiments, the modified gRNAs exhibit increased resistance to nucleases. In some embodiments, a gRNA comprises two separate RNA molecules (i.e., a “dual gRNA”). A dual gRNA comprises two separate RNA molecules: a “crispr RNA” (or “crRNA”) and a “tracr RNA” and is well known to one of skill in the art.

Generally, various gene editing technologies are known, which can be applied according to various embodiments of this disclosure. Gene editing technologies include but are not limited to zinc fingers (ZFs), transcription activator-like effectors (TALEs), etc. Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducted using CRISPR associated Cas system (e.g., CRISPR-Cas9), as known in the art. See, for example, U.S. Pat. Nos. 8,697,359, 8,906,616, and 8,999,641, each of which is hereby incorporated by reference in its entirety. In various embodiments, the gene editing employs a Type II Cas endonuclease (such as Cas9) or employs a Type V Cas endonuclease (such as Cas12a). Type II and Type V Cas endonucleases are guide RNA directed. Design of gRNAs to guide the desired gene edit (while limiting or avoiding off target edits) is known in the art. See, for example, Mohr S E, et al., CRISPR guide RNA design for research applications, FEBS J. 2016 September; 283(17): 3232-3238. In still other embodiments, non-canonical Type II or Type V Cas endonucleases having homology (albeit low primary sequence homology) to S. pyogenes Cas9 or Prevotella and Francisellal (Cpf1 or Cas12a) can be employed. Numerous such non-canonical Cas endonucleases are known in the art. Nidhi S, et al. Novel CRISPR-Cas Systems: An Updated Review of the Current Achievements, Applications, and Future Research Perspectives, Int J Mol Sci. 2021 April; 22(7): 3327. In still other embodiments, the gene editing employs base editing or prime editing to incorporate mutations without instituting double strand breaks. See, for example, Antoniou P, et al., Base and Prime Editing Technologies for Blood Disorders, Front. Genome Ed., 28 Jan. 2021; Matsuokas IG, Prime Editing: Genome Editing for Rare Genetic Diseases Without Double-Strand Breaks or Donor DNA, Front. Genet., 9 Jun. 2020. Various other gene editing processes are known, including use of dead Cas (dCas) systems (e.g., Cas fusion proteins) to target DNA modifying enzymes to desired targets using the dCas as a guide RNA-directed system. Brezgin S, Dead Cas Systems: Types, Principles, and Applications, Int J Mol Sci. 2019 December; 20(23): 6041.

Base editors that can install precise genomic alterations without creating double-strand DNA breaks can also be used in gene editing (e.g., designing gene therapy vectors) in the cells (e.g., iPSCs). Base editors essentially comprise a catalytically disabled nuclease, such as Cas9 nickase (nCas9), which is incapable of making DSBs and is fused to a nucleobase deaminase enzyme and, in some cases, a DNA glycosylase inhibitor. Currently, there are 2 major categories of base editors, cytidine base editors (CBEs) and adenine base editors (ABEs), which catalyze C>T and A>G transitions. Base editors can be delivered, for example, via HDAd5/35++ vectors to efficiently edit promoters and enhancers to active or inactivate a gene. Exemplary methods are described in U.S. Pat. Nos. 9,840,699; 10,167,457; 10,113,163; 11,306,324; 11,268,082; 11,319,532; and 11,155,803. Also contemplated are prime editors that comprise a reverse transcriptase conjugated to (e.g., fused with) a Cas endonuclease and a polynucleotide useful as a DNA synthesis template conjugated to (e.g., fused with) a guide RNA, as described in WO 2020/191153.

Exemplary vectors that can be used for the genome editing applications include, but are not limited to, plasmids, retroviral vectors, lentiviral vectors, adenovirus vectors (e.g., Ad5/35, Ad5, Ad26, Ad34, Ad35, Ad48, parvovirus (e.g., adeno-associated virus (AAV) vectors, herpes simplex virus vectors, baculoviral vectors, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., canarypox, vaccinia or modified vaccinia virus. The vector comprising the nucleic acid molecule of interest may be delivered to the cell (e.g., iPS cells, endothelial cells, hemogenic endothelial cells, HSCs (ST-HSCs or LT-HSCs) via any method known in the art, including but not limited to transduction, transfection, infection, and electroporation. Any of these vectors may include transposable element (such as a piggyback transposon or sleeping beauty transposon). Transposons insert specific sequences of DNA into genomes of vertebrate animals. The gene of interest can be integrated into the genome of a mammalian cell by transposase-catalyzed cleavage of similar excision sites that exist within the nuclear genome of the cell.

For increased efficiency, in some embodiments, the Cas and the gRNA can be combined before being delivered into the cells. The Cas-gRNA complex is known as a ribonucleoprotein (RNP). A number of methods have been developed for direct delivery of RNPs to cells. For example, RNP can be delivered into cells in culture by lipofection or electroporation. Electroporation using a nucleofection protocol can be employed, and this procedure allows the RNP to enter the nucleus of cells quickly, so it can immediately start cutting the genome. See, for example, Zhang S, Shen J, Li D, Cheng Y. Strategies in the delivery of Cas9 ribonucleoprotein for CRISPR/Cas9 genome editing. Theranostics. 2021 Jan. 1; 11(2):614-648, hereby incorporated by reference in its entirety. In some embodiments, Cas9 and gRNA are electroporated as RNP into the donor iPSCs and/or HSCs.

Generally, a protospacer adjacent motif (PAM) is required for a Cas nuclease to cut and is generally found 3-4 nucleotides downstream from the cut site. The PAM is a short DNA sequence (usually 2-6 base pairs in length) that follows the DNA region targeted for cleavage by the CRISPR system, such as CRISPR-Cas9. In some embodiments, the PAM sequences, sgRNAs, or base editing tools targeting haplotypes or polymorphs of HLA loci does not include four Gs, four Cs, GC repeats, or combinations thereof.

In some embodiments, a CRISPR/Cas9 system specific to a unique HLA haplotype can be developed by designing singular gRNAs targeting each of the donor-specific HLA-A, HLA-DPB1, and HLA-DQB1 genes (for example), using the gRNAs as described herein. To perform genetic knockout, the gRNA targets the Cas9 protein to the appropriate site to edit. Next, the Cas9 protein can perform a double strand break (DSB), where the DNA repairs through a non-homologous end joining (NHEJ) mechanism which generates indels resulting in a frameshift mutation and terminates the resulting protein's function. However, off-target genetic modifications can occur and alter the function of otherwise intact genes. For example, the Cas9 endonuclease can create DSBs at undesired off-target locations, even in the presence of some degree of mismatch. This off-target activity can create genome instability events, such as point mutations and genomic structural variations. In various embodiments, a sgRNA targeting HLA-A can target a region of chromosome 6 defined as 29942532-29942626. In various embodiments, a sgRNA targeting HLA-DQB1 can target a region of chromosome 6 defined as 32665067-32664798. In various embodiments, a sgRNA targeting HILA-DPB1 can target a region of chromosome 6 defined as 33080672-33080935.

gRNAs can be used to develop clonal iPSCs. Such iPSC lines can be evaluated for (i) ON-target edits, (ii) OFF-target edits, and (iii) Translocation edits, for example using sequencing, as described herein. Specifically, such assays can be performed by multiplex PCR with primers designed to target and enrich regions of interest followed by next-generation sequencing (e.g., Amplicon sequencing, AMP-seq). The ON-target panel and the translocation panel can amplify the intended edited region, allowing for selection of iPSC clones with the expected edits which are free from chromosomal translocation arising from unintended DSB cut-site fusion. The OFF-target panel can enrich any potential off-target regions identified via sequencing and allows for selection of iPSC clones with negligible off-target mutations. Together, these assays enable a screen of the iPSC clones to select the clones with the desired edits, while excluding potential CRISPR/Cas9-related genome integrity issues.

In some embodiments, to further ensure the genomic stability and integrity of reprogrammed and edited iPSCs, genetic and genomic assays can be performed to select for clones which, for example, did not undergo translocation and mutation events, and that did not integrate the episomal vectors. For example, whole-genome sequencing (WGS) is performed on CD34+ cells and on iPSC clones after reprogramming, where the genomes are compared for differences arising from editing. These analyses provide an assessment of which iPSC clone genomes differ from the CD34+ starting material, enabling informed selection iPSC clones which did not accrue mutations during the reprogramming.

In some embodiments, karyotyping analyses using systems such as KARYOSTAT assays is used to select iPSC clones which did not accrue indels and translocation during the reprogramming, for example as described in Ramme A P, et al, “Supporting dataset of two integration-free induced pluripotent stem cell lines from related human donors,” Data Brief. 2021 May 15; 37:107140, hereby incorporated by reference in its entirety. KARYOSTAT assays allow for visualization of chromosome aberrations with a resolution similar to G-banding karyotyping. The size of structural aberration that can be detected is >2 Mb for chromosomal gains and >1 Mb for chromosomal losses. The KARYOSTAT array is functionalized for balanced whole-genome coverage with a low-resolution DNA copy number analysis, where the assay covers all 36,000 RefSeq genes, including 14,000 OMIM targets. The assay enables the detection of aneuploidies, submicroscopic aberrations, and mosaic events.

In some embodiments, Array Comparative Genomic Hybridization (aCGH) analyses is used to select iPSC clones which did not accrue copy number aberrations (CNA) during reprogramming, for example as described in Wiesner et al. “Molecular Techniques,” Editor(s): Klaus J. Busam, Pedram Gerami, Richard A. Scolyer, “Pathology of Melanocytic Tumors,” Elsevier, 2019, pp. 364-373, ISBN 9780323374576; and Hussein S M, et al. “Copy number variation and selection during reprogramming to pluripotency,” Nature. 2011 Mar. 3; 471(7336):58-62, hereby incorporated by reference in its entirety. aCGH is a technique that analyzes the entire genome for CNA by comparing the sample DNA to reference DNA.

In some embodiments, targeted heme malignancy NGS panel analyses is used to select iPSC clones which did not accrue hematologic malignancy mutations during reprogramming. For example, targeted heme malignancy NGS panels can focus on myeloid leukemia, lymphoma, and/or other hematologic malignancy-associated genes to generate a smaller, more manageable data set than broader methods. Targeted heme malignancy NGS panel analysis includes the use of highly multiplexed PCR to amplify regions associated with hematologic malignancies followed by next-generation sequencing.

In some embodiments, Droplet Digital PCR (ddPCR) is used to select iPSC clones which did not integrate episomal vectors and that have been passaged enough for episomal vector clearance. As discussed herein, iPSC reprogramming of CD34+ cells can be achieved by delivering episomal vectors encoding reprogramming factors. However, episomal vectors can, albeit rarely, randomly integrate into the cellular genome, which could disrupt developmental processes, homeostasis, etc. Therefore, ddPCR methods can be used to detect residual episomal vector in the iPSC cultures and enable selection of iPSC clones which did not integrate episomal vectors.

In some embodiments, after assessing that the selected clones are free from genomic aberrations related to editing, the clones can be additionally tested for spontaneous mutations that might arise during expansion. For example, mutations affecting hematologic malignancy genes, indel, translocations, number aberrations, e.g., as described for the pre-edited reprogrammed clones. Analyses for spontaneous mutations can include whole-genome sequencing (WGS), KARYOSTAT analysis, Array Comparative Genomic Hybridization (aCGH) analysis, targeted heme malignancy NGS panel AMP-Seq analysis, and/or Droplet Digital PCR (ddPCR).

Somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and kWf4. In some embodiments, the reprogramming factors are Sox2, Oct3/4, c-Myc, and klf4. In some embodiments reprogramming factors comprise Oct-4, Sox-2, Klf-4, 1-Myc, Lin-28, SV40 Large T Antigen (“SV40LT”), and short hairpin RNAs targeting p53 (“shRNA-p53”). Methods for preparing iPSCs are described, for example, in U.S. Pat. Nos. 10,676,165; 9,580,689; 10,221,395, and 9,376,664, which are hereby incorporated by reference in their entireties. In various embodiments, reprogramming factors are expressed using well known viral vector systems, such as lentiviral, Sendai, or measles viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations. In some embodiments, the method comprises reprogramming CD36+ basophilic erythroblasts for pluripotency using a vector (e.g., viral or episomal) expressing POU5F1/OCT4, SOX2, KLF4, and c-MYC as a polycistronic unit. In some embodiments, the method comprises reprogramming PBMCs with Oct4, Sox2, Lin28, Klf4 and L-myc.

In some embodiments, the human pluripotent stem cells (e.g., iPSCs) are gene-edited. Gene-editing can include, but is not limited to, modification of HLA genes (e.g., deletion of one or more HLA Class I and/or Class II genes), deletion of P2 microglobulin (P2M), and deletion of CIITA.

In some embodiments, the iPSCs of the present disclosure are gene edited to encode a hyperresponsive EPO Receptor. Erythropoiesis is the process for production of red blood cells. Erythropoietin (EPO) is the key hormone responsible for effective erythropoiesis. Erythropoietin receptor (EPOR) is a protein that in humans is encoded by the EPOR gene. EPOR is a 52 kDa peptide with a single carbohydrate chain resulting in an approximately 56-57 kDa protein found on the surface of EPO responding cells. The most well-established function of EPOR is to promote proliferation and rescue of erythroid (red blood cell) progenitors from apoptosis.

Beneficial mutations in the EPOR have been reported, where increased red blood cell number allows for improved oxygen delivery in athletic endurance events with no apparent adverse effects upon the athlete's health. de La Chapelle et al., PNAS 1993; 90.10:4495-4499. Generally, truncating mutations removing only parts of the intracellular EPOR C-terminus that bind negative regulators have been reported to associate with primary erythrocytosis, with low EPO and high hemoglobin levels. These mutations make EPOR hyper-responsive to EPO with a secondary effect of increasing hemoglobin levels. See Juvonen, E., Ikkala, E., Fyhrquist, F. & Ruutu, T. Autosomal dominant erythrocytosis caused by increased sensitivity to erythropoietin. Blood 1991; 78, 3066-3069. SHP-1 is known to play an important role in the signal transduction of EPOR, by associating via its SH2 domains to the receptor and dephosphorylating key substrates. Jiao, H., Berrada, K., Yang, W., Tabrizi, M., Platanias, L. C., & Yi, T. Direct association with and dephosphorylation of Jak2 kinase by the SH2-domain-containing protein tyrosine phosphatase SHP-1. Molecular and Cellular Biology, 1996; 16(12), 6985-6992.

Thus, in various embodiments, the HSC populations of the present disclosure express an EPOR having a truncating mutation. In some embodiments, the HSC populations of the present disclosure express an EPOR lacking or having a mutated SHP-1 inhibitory domain. In some embodiments, the HSC populations of the present disclosure express an EPOR having one or more missense or frameshift mutations that result in hyperresponsiveness, optionally by mutation or deletion of the SHP-1 inhibitory domain.

These HSC populations can be used for more efficient ex vivo red cell production, or in other embodiments, can be used to deliver HSCs or erythroid progenitors to patients in need to reduce or eliminate requirements for periodic blood transfusions. Further, because such cells can be gene edited to delete certain HLA genes (as described), the HSC populations and erythroid progenitors can be easily HLA-matched for a recipient. HSCs according to the disclosure differentiate to various hematopoietic lineages (similar to bone marrow CD34+ cells), and are capable of restoring hematopoietic system in a recipient.

In various embodiments, iPSCs are prepared, and expanded using a conventional culture system. Expanded iPSCs can be recovered from the culture for generating embryoid bodies (EBs). EBs, created by differentiation of iPSCs, are three-dimensional aggregates of iPSCs and comprise the three (or alternatively two or one) embryonic germ layer(s) based on the differentiation method(s). Preparation of EBs is described, for example, in US 2019/0177695, which is hereby incorporated by reference in its entirety. In some embodiments, EBs prepared by differentiation of the iPSCs, are expanded in a bioreactor as described, for example, in Abecasis B. et al., Expansion of 3D human induced pluripotent stem cell aggregates in bioreactors: Bioprocess intensification and scaling-up approaches. J of Biotechnol. 246 (2017) 81-93. EBs can be used to generate any desired cell type. Other methods, including a 3D suspension culture, for expansion or differentiation of EBs is described in WO 2020/086889, which is hereby incorporated by reference in its entirety.

In some embodiments, the process according to each aspect can comprise generating CD34+ enriched cells from the pluripotent stem cells (e.g., EBs) and inducing endothelial-to-hematopoietic differentiation. HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means.

In some embodiments, the method comprises preparing endothelial cells with hemogenic potential from pluripotent stem cells, prior to induction of EHT. In some embodiments, the method comprises overexpression of E26 transformation-specific variant 2 (ETV2) transcription factor in the iPSCs. ETV2 can be expressed by introduction of an encoding non-integrating episomal plasmid, for constitutive or inducible expression of ETV2, and for production of transgene-free hemogenic ECs. In some embodiments, ETV2 is expressed from an mRNA introduced into the iPSCs. mRNA can be introduced using any available method, including electroporation or lipofection. Differentiation of cells expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). Cells generated in this manner may be used for producing CD34+ cells and inducing EHT according to embodiments of this disclosure.

Following CD34+ enrichment, HSCs are then generated from the endothelial cells using mechanical, biochemical, pharmacological and/or genetic stimulation or modification.

In some embodiments, iPSC differentiation proceeds until cells are at least about 10% CD34+, or at least about 20% CD34+, or at least about 25% CD34+, or at least about 30% CD34+. In some embodiments, CD34 enrichment and EHT may be induced at Day 7 to Day 14 of iPSC differentiation, such as for example, Day 8, Day 9, Day 10, Day 11, Day 12, Day 13, or Day 14. In some embodiments, CD34+ cells are harvested at about Day 8. Differentiation of iPSCs can be according to known techniques. In some embodiments, iPSC differentiation involves factors such as, but not limited to, combinations of bFGF, Y27632, BMP4, VEGF, SCF, EPO, TPO, IL-6, IL-11, and/or IGF-1. In some embodiments, hPSCs are differentiated using feeder-free, serum-free, and/or GMP-compatible materials, such as pomalidomide or lenalidomide. In some embodiments, hPSCs are co-cultured with murine bone marrow-derived feeder cells such as OP9 or MS5 cell line in serum-containing medium. The culture can contain growth factors and cytokines to support differentiation of embryoid bodies or monolayer system. The OP9 co-culture system can be used to generate multipotent HSPCs, which can be differentiated further to several hematopoietic lineages including T lymphocytes, B lymphocytes, megakaryocytes, monocytes or macrophages, and erythrocytes. See Netsrithong R. et al., Multilineage differentiation potential of hematoendothelial progenitors derived from human induced pluripotent stem cells, Stem Cell Research & Therapy Vol. 11 Art. 481 (2020). Alternatively, a step-wise process using defined conditions with specific signals can be used. For example, the expression of HOXA9, ERG, RORA, SOX4, and MYB in human PSCs favors the direct differentiation into CD34+/CD45+ progenitors with multilineage potential. Further, expression of factors such as HOXB4, CDX4, SCL/TAL1, or RUNX1a support the hematopoietic program in human PSCs. See Doulatov S. et al., Induction of multipotential hematopoietic progenitors from human pluripotent stem cells via re-specification of lineage-restricted precursors, Cell Stem Cell. 2013 Oct. 3; 13(4).

Induction of EHT can be with any known process. In some embodiments, induction of EHT generates a hematopoietic stem cell (HSC) population comprising LT-HSCs. In some embodiments, EHT generates HSCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and hematopoietic stem progenitor cells.

In some embodiments, the method comprises increasing the expression or activity of dnmt3b in PSCs, embryoid bodies, CD34+-enriched cells, ECs, HECs or HSCs, which can be by mechanical, genetic, biochemical, or pharmacological means. In some embodiments, the method comprises increasing activity or expression of DNA (cytosine-5-)-methyltransferase 3 beta (Dnmt3b) and/or GTPase IMAP Family Member 6 (Gimap6) in the cells. See WO 2019/236943 and WO 2021/119061, which is hereby incorporated by reference in its entirety. In some embodiments, the induction of EHT comprises increasing the expression or activity of dnmt3b.

In some embodiments, cells are contacted with an effective amount of an agonist of a mechanosensitive receptor or a mechanosensitive channel that increases the activity or expression of Dnmt3b. In some embodiments, the mechanosensitive receptor is Piezol. An exemplary Piezol agonist is Yoda1. In some embodiments, the mechanosensitive receptor is Trpv4. An exemplary Trpv4 agonist is GSK1016790A. Yodal (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezol. eLife (2015). Yoda 1 has the following structure:

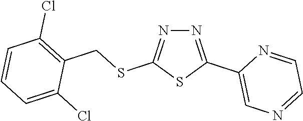 [SMILES: Clc1cccc(Cl)c1CSc1nnc(-c2cnccn2)s1]

Derivatives of Yodal can be employed in various embodiments. For example, derivatives comprising a 2,6-dichlorophenyl core are employed in some embodiments. Exemplary agonists are disclosed in Evans E L, et al., Yodal analogue (Dookul) which antagonizes Yodal-evoked activation of Piezol and aortic relaxation, British J. of Pharmacology 175(1744-1759): 2018. Still other Piezol agonist include Jedi1, Jedi2, single-stranded (ss) RNA (e.g., ssRNA40), and derivatives and analogues thereof. See Wang Y., et al., A lever-like transduction pathway for long-distance chemical-and mechano-gating of the mechanosensitive Piezol channel Nature Communications (2018) 9:1300; Sugisawa, et al., RNA Sensing by Gut Piezol Is Essential for Systemic Serotonin Synthesis, Cell, Volume 182, Issue 3, 2020, Pages 609-624, incorporated herein in their entirety by reference. These Piezol agonists are commercially available. In various embodiments, the effective amount of the Piezol agonist or derivative is in the range of about 1 μM to about 500 βM, or about 5 μM to about 200 μM, or about 5 M to about 100 μM, or in some embodiments, in the range of about 25 μM to about 150 μM, or about 25 μM to about 100 βM, or about 25 μM to about 50 μM.

In various embodiments, pharmacological Piezol activation is applied to CD34+ cells (i.e., CD34-enriched cells). In certain embodiments, pharmacological Piezol activation may further be applied to iPSCs, embryoid bodies, ECs, hemogenic endothelial cells (HECs), HSCs, hematopoietic progenitors, as well as hematopoietic lineage(s). In certain embodiments, Piezol activation is applied at least to one or more of iPSCs, EBs generated from iPSCs, or CD34+ cells isolated from EBs, and/or combinations thereof, which in accordance with various embodiments, allows for superior generation of erythroid progenitor cells as compared to other methods for inducing EHT.

In some embodiments, Piezol activation is not used during induction of EHT.

In certain embodiments, expansion of hematopoietic cells may also include culturing the cells in contact with an immunomodulatory compound, e.g., a TNF-alpha inhibitory compound, for a time and in an amount sufficient to cause a detectable increase in the proliferation of the hematopoietic cells over a given time, compared to an equivalent number of hematopoietic cells not contacted with the immunomodulatory compound. See, e.g., U.S. Pat. No. 7,198,171, the disclosure of which is hereby incorporated by reference in its entirety. In an embodiment, the immunomodulatory compound is 3-(4-amino-1-oxo-1,3-dihydroisoindol-2-yl)-piperidine-2,6-dione; 3-(4′aminoisoindolin-1-one)-I-piperidine-2,6-dione; 4-(amino)-2-(2,6-dioxo(3-piperidyl))-isoindoline-1,3-dione; 4-amino-2-[(3RS)-2,6-dioxopiperidin-3-yl]-2H-isoindole-1,3-dione; ca-(3-aminophthalimido) glutarimide; pomalidomide, lenalidomide, or thalidomide.

Alternatively, or in addition, the activity or expression of Dnmt3b can be increased directly in the cells, e.g., in CD34-enriched cells. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the cells, such as, but not limited to, to increase promoter strength, ribosome binding, RNA stability, and/or impact RNA splicing.

In some embodiments, the method comprises increasing the activity or expression of Gimap6 in the cells, alone or in combination with Dnmt3b and/or other genes that are up- or down regulated upon cyclic strain or Piezol activation. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene. In some embodiments, gene editing is employed to introduce a genetic modification to Gimap6 expression elements in the cells (such as one or more modifications to increase promoter strength, ribosome binding, RNA stability, or to impact RNA splicing).

In embodiments of this disclosure employing mRNA delivery to cells, known chemical modifications can be used to avoid the innate-immune response in the cells. For example, synthetic RNA comprising only canonical nucleotides can bind to pattern recognition receptors and can trigger a potent immune response in cells. This response can result in translation block, the secretion of inflammatory cytokines, and cell death. RNA comprising certain non-canonical nucleotides can evade detection by the innate immune system and can be translated at high efficiency into protein. See U.S. Pat. No. 9,181,319, which is hereby incorporated by reference, particularly with regard to nucleotide modification to avoid an innate immune response.

In some embodiments, expression of Dnmt3b and/or Gimap6 is increased by introducing a transgene into the cells, which can direct a desired level of overexpression (with various promoter strengths or other selection of expression control elements). Transgenes can be introduced using various viral vectors or transfection reagents (including Lipid Nanoparticles) as are known in the art. In some embodiments, expression of Dnmt3b and/or Gimap6 is increased by a transgene-free method (e.g., episome delivery). In some embodiments, expression or activity of Dnmt3b and/or Gimap6 or other genes disclosed herein are increased using a gene editing technology, for example, to introduce one or more modifications to increase promoter strength, ribosome binding, or RNA stability.

In some embodiments, the method comprises applying cyclic 2D, 3D, or 4D stretch to cells. In various embodiments, the cells subjected to cyclic 2D, 3D, or 4D stretch are selected from one or more of CD34-enriched cells, iPSCs, ECs, and HECs. For example, a cell population is introduced to a bioreactor that provides a cyclic-strain biomechanical stretching, as described in WO 2017/096215, which is hereby incorporated by reference in its entirety. The cyclic-strain biomechanical stretching can increase the activity or expression of Dnmt3b and/or Gimap6. In these embodiments, mechanical means apply stretching forces to the cells, or to a cell culture surface having the cells (e.g., ECs or HECs) cultured thereon. For example, a computer controlled vacuum pump system or other means for providing a stretching force (e.g., the FlexCell™ Tension System, the Cytostretcher System) attached to flexible biocompatible and/or biomimetic surface can be used to apply cyclic 2D, 3D, or 4D stretch ex vivo to cells under defined and controlled cyclic strain conditions. For example, the applied cyclic stretch can be from about 1% to about 20% cyclic strain (e.g., about 6% cyclic strain) for several hours or days (e.g., about 7 days). In various embodiments, cyclic strain is applied for at least about one hour, at least about two hours, at least about six hours, at least about eight hours, at least about 12 hours, at least about 24 hours, at least about 48 hrs, at least about 72 hrs, at least about 96 hrs, at least about 120 hrs, at least about 144 hrs, or at least about 168 hrs.

Alternatively, or in addition, EHT is stimulated by Trpv4 activation. The Trpv4 activation can be by contacting cells (e.g., CD34-enriched cells, ECs, or HECs) with one or more Trpv4 agonists, which are optionally selected from GSK1016790A, 4alpha-PDD, or analogues and/or derivatives thereof.

Where cell populations are described herein as having a certain phenotype it is understood that the phenotype represents a significant portion of the cell population, such as at least 25%, at least 40%, or at least about 50%, or at least about 60%, or at least about 75%, or at least about 80%, or at least about 90% of the cell population. Further, at various steps, cell populations can be enriched for cells of a desired phenotype, and/or depleted of cells of an undesired phenotype, such that cell population comprise at least about 75%, or at least about 80%, or at least about 90% of the desired phenotype. Such positive and negative selection methods are known in the art. For example, cells can be sorted based on cell surface antigens (including those described herein) using a fluorescence activated cell sorter, or magnetic beads which bind cells with certain cell surface antigens. Negative selection columns can be used to remove cells expressing undesired cell-surface markers. In some embodiments, cells are enriched for CD34+ cells (prior to and/or after undergoing EHT). In some embodiments, the cell population is cultured under conditions that promote expansion of CD34+ cells to thereby produce an expanded population of stem cells.

In various embodiments, CD34+ cells (e.g., the floater and/or adherent cells) are harvested from the culture undergoing endothelial-to-hematopoietic transition. In various embodiments, the HSCs or CD34-enriched cells are further expanded. For example, the HSCs or CD34-enriched cells can be expanded according to methods disclosed in U.S. Pat. Nos. 8,168,428; 9,028,811; 10,272,110; and 10,278,990, which are hereby incorporated by reference in their entireties. In some embodiments, ex vivo expansion of HSCs or CD34-enriched cells employs prostaglandin E2 (PGE2) or a PGE2 derivative. In some embodiments of this disclosure, the HSCs comprise at least about 0.01% LT-HSCs, or at least about 0.05% LT-HSCs, or at least about 0.1% LT-HSCs, or at least about 0.5% LT-HSCs, or at least about 1% LT-HSCs.

Hematopoietic stem cells (HSCs) which give rise to erythroid, myeloid, and lymphoid lineages, can be identified based on the expression of CD34+ cells and the absence of lineage specific markers (termed Lin−). In some embodiments, a population of stem cells comprising HSCs are enriched, for example, as described in U.S. Pat. No. 9,834,754, which is hereby incorporated by reference in its entirety. For example, this process can comprise sorting a cell population based on expression of one or more of CD34, CD90, CD38, and CD43 (HSC markers) or CD71 (marker for early erythroid progenitors) or CD235a (marker for mature erythroid progenitors). A fraction can be selected for further differentiation that is one or more of CD34+, CD90+, CD38−, and CD43−. In some embodiments, the stem cell population for differentiation to a hematopoietic lineage is at least about 80% CD34+, or at least about 90% CD34+, or at least about 95% CD34+.

In some embodiments, the stem cell population, or CD34-enriched cells or fraction thereof, or derivative population are expanded as described in US 2020/0308540, which is hereby incorporated by reference in its entirety. For example, the cells are expanded by exposing the cells to an aryl hydrocarbon receptor antagonist including, for example, SR1 or an SR1-derivative. See also, Wagner et al., Cell Stem Cell 2016; 18(1):144-55 and Boitano A., et al., Aryl Hydrocarbon Receptor Antagonists Promote the Expansion of Human Hematopoietic Stem Cells. Science 2010 Sep. 10; 329(5997): 1345-1348.

In some embodiments, the compound that promotes expansion of CD34+ cells include a pyrimidoindole derivative including, for example, UM171 or UM729 (see US 2020/0308540, which is hereby incorporated by reference).

In some embodiments, the stem cell population or CD34-enriched cells are further enriched for cells that express Periostin and/or Platelet Derived Growth Factor Receptor Alpha (pdgfra) or are modified to express Periostin and/or pdgfra, as described in WO 2020/205969 (which is hereby incorporated by reference in its entirety). Such expression can be by delivering encoding transcripts to the cells, or by introducing an encoding transgene, or a transgene-free method, not limited to introducing a non-integrating episome to the cells. In some embodiments, gene editing is employed to introduce a genetic modification to expression elements in the cells, such as to modify promoter activity or strength, ribosome binding, RNA stability, or impact RNA splicing.

In still other embodiments, the stem cell population or CD34-enriched cells are cultured with an inhibitor of histone methyltransferase EZH1. Alternatively, EZH1 is partially or completely deleted or inactivated or is transiently silenced in the stem cell population. Inhibition of EZH1 can direct myeloid progenitor cells (e.g., CD34+CD45+) to erythroblast lineages. In still other embodiments, EZH1 is overexpressed in the stem cell population.

In some embodiments, the HSC population or fraction thereof is differentiated to red cells or progenitors or derivatives thereof. For example, the HSC population (or cells isolated therefrom) is cultured with EPO, IZL-3 and SCF, for example, (and/or other extracellular matrix component(s)), and/or combinations thereof, to produce a population comprising erythroid progenitor cells or a derivative cell population (e.g., erythrocytes). The HSC population of cells give rise to high percentage of burst forming unit-erythroid (BFU-E) cells, and colony forming units-erythroid (CFU-E) cells, which are indications of induction of erythropoiesis. These cells may be further enriched and/or expanded.

For example, the production of iPS cell lines from peripheral blood samples can comprise the initial following steps: erythroblast enrichment, and iPSC initiation. In the erythroblast enrichment step, the cells are reprogrammed, for example with POU5F1/OCT4, SOX2, KLF4, and c-MYC transfection (or as otherwise described herein). In some embodiments, the erythroblast population exceeds 80% after enrichment. Once the pluripotency of the iPSC cells is confirmed by the presence of pluripotency markers, such as but not limited to, POU5F1/OCT4, SOX2, LIN28, KLF4 AND NANOG, reprogrammed iPSCs are cultured under culture condition to generate embryoid bodies (EB). CD34+ cells are isolated/enriched from dissociated EBs between Day 8 and 14 of iPSC differentiation (as already described). After inducing EHT in CD34+ cells (optionally followed by further enrichment of CD34+ cells and/or enrichment with other erythroid progenitor markers), the cells are cultured under conditions for erythroid differentiation.

In some embodiments, the HSC population or fraction thereof is differentiated to erythroid cells or progenitors or derivatives thereof independent of the use of an agonist of a mechanosensitive receptor or a mechanosensitive channel such as Yodal. In some embodiments, the use of an agonist of a mechanosensitive receptor or a mechanosensitive channel such as Yodal is optional. Thus, in some embodiments, CD34+ cells are enriched from a differentiated pluripotent stem cell population to prepare a CD34+-enriched population. Endothelial-to-hematopoietic transition of the CD34+-enriched cell population is induced for at least two days, but no more than 12 days in which the use of an agonist of a mechanosensitive receptor or a mechanosensitive channel such as Yodal, jedi1, jedi2, ssRNA40 is optional. The HSCs and/or HSPCs are differentiated to a progenitor erythroid cell population or an erythroid cell population.

In some embodiments, the endothelial-to-hematopoietic transition of the CD34+-enriched cell population is induced for at least for two days and optionally further for at least about 4 hours, or at least about 8 hours, or at least about 12 hours, or at least about 16 hours, or at least about 20 hours, or at least about 24 hours, or at least about 2 days, or at least about 3 days, or at least about 4 days, or at least about 5 days, or at least about 6 days, or at least about 7 days, or at least about 8 days, or at least about 9 days, or at least about 10 days. Generally, EHT is not induced for more than 12 days.

During differentiation, one can assess, for example, the proliferative progenitor stages of the erythroid committed cells by assessing their colony formation capabilities in semisolid media. For example, harvested cells can give rise to hematopoietic colonies with a high percentage of burst-forming unit-erythroid (BFU-E) and colony-forming unit erythroid (CFU-E) cells. The Hb content of the cells can generally be used to define their developmental stage, for example by evaluating the γ to β globin switch in vitro.

In some embodiments, this process makes use of serum-free, xeno-free protocol which is compatible with Good Manufacturing Practice (GMP). In some embodiments, this process makes use of feeder layer such as OP9 or coculturing with other cells, such as stromal cells as an example. In some embodiments, this process makes use of small molecules, such as StemRegenin (SR1, a dual RasGAP and ERK1/2 inhibitor), Yodal, Jedi1, Jedi2, ssRNA 40 or analogues or derivatives thereof, BIO (archetypal GSK3b inhibitor), CHIR99021 (GSK3b inhibitor), IBMX (nonspecific inhibitor of cAMP and cGMP phosphodiesterases), and A-A014418 (GSK3b inhibitor VIII) as substitutes for growth factors or various cytokines to reduce side effects and media costs.

Use of bioreactors, modifying microenvironments using macrophages and small molecules, and utilizing genetic alterations to enhance survival of mature RBCs, increase enucleation rate, and promote hemoglobin switching are also contemplated in various embodiments and aspects of the invention. As an example of the use of a bioreactor to generate enucleated cells, the HSCs or erythroid progenitor cells can be cultured in a bioreactor under one or more maturation conditions. Maturation conditions can include: (i) a predetermined pH; (ii) a predetermined or specific level of (dissolved) oxygen; and (iii) mechanical stress.

The predetermined pH may be selected from a pH of about 4.0 to about 7.9. For example the maturation conditions may comprise a pH in the range of about 5.0 to about 7.9, or about 6.0 to about 7.9, or about 7.0 to about 7.9 (such as about 7.4 to about 7.5). The maturation conditions might comprise one or more of the above listed pH values; for example, the maturation conditions may modulate between different pH levels—for example a first pH and a second pH.

The maturation conditions may exploit a level of oxygen which is less than about 90%, or in other embodiments less than about 80%, or less than about 70%, or less than about 60%, or less than about 50%, or less than about 40% or less than about 30% of atmospheric oxygen. For example, the methods of this invention may exploit a level of oxygen which is less than about 50% of atmospheric oxygen (e.g., from about 25% to about 50% of atmospheric oxygen). The maturation conditions may exploit a level of dissolved oxygen (i.e. oxygen dissolved in the culture medium) of from about 2% to about 29%, such as from about 5% to about 20%, or from about 5% to about 15% (e.g., about 11%).

A level of mechanical stress may be created controlling the speed at which a bioreactor impeller tip moves through a cell culture. Impeller tip speeds of about 50 to about 500 rpm, for example speeds of about 100 to about 450 rpm, or about 150 to about 300 rpm or about 200 to about 250 rpm may be used. The level of mechanical stress used may modulate between two or more predetermined levels of mechanical stress. Under such circumstances, and where a bioreactor is used to impart the necessary mechanical stress, variable or modulating mechanical stress used may be created via the selection (and use) of one or more different impeller speeds. Level or levels of mechanical stress may be applied for any suitable period of time. For example, a level or levels of mechanical stress may be applied for several minutes (e.g., 10 to 60 minutes), one or more hours (e.g., one to ten hours), or one or more days (e.g., one to ten days). For example, a level or levels of mechanical stress may be applied continuously or intermittently throughout the period.

In an embodiment, an erythroid expansion medium at least comprising EPO and optionally comprising cytokines and growth factors is used, which can be supplemented with one or more of small molecule compounds selected from (i) a piezol agonist, (ii) a Trpv4 agonist, (iii) phosphodiesterase inhibitor or (iv) GSK 3 inhibitor, including compounds and concentrations described herein.

Another critical issue for clinical utilization of hESC-derived RBCs is whether they can be enucleated in vitro. Under conditions disclosed herein, RBCs undergo differentiation events, including a progressive decrease in size and increase in glycophorin A expression (a mature RBC marker) and chromatin/nuclear condensation, which results in the extrusion of the pyCnotic nucleus to form enucleated erythrocytes with a diameter of 6 to 8 μm, which is similar to normal RBCs.

Events associated with enucleation, can be assessed by examining multiple characteristics related to the process of erythrocyte maturation. For example, a progressive decrease in cell size and the nuclear-to-cytoplasmic ratio (N/C) ratio before enucleation occurs, the size and N/C of these cells decrease significantly overtime indicating substantial nuclear condensation during the process. Also, during this process cells express a high level of CD71, an early erythroblast marker, and decrease their expression over time, although they show low to negligible level of CD235a (glycophorin A) protein, a mature erythrocyte marker, in the beginning, but increase their expression dramatically with their maturation. Benzidine stains can be used to show a progressive accumulation of hemoglobin in the cells and a decrease in cell size over time.

In some embodiments, the erythrocytes or the red blood cells or precursors thereof (e.g., iPSCs) can be genetically engineered so that they can carry a wide variety of useful cargoes to specific locations in the body.

In other aspects, the invention provides a red cell or erythroid lineage population, or pharmaceutically acceptable composition thereof, produced by the method described herein. In various embodiments, the composition comprises the desired cell population (e.g., erythrocytes) and a pharmaceutically acceptable vehicle. The pharmaceutical composition may comprise at least about 10⁵, or at least about 10⁶, or at least about 10⁷ erythrocytes per mL. The pharmaceutical composition may be provided in units of about 50 mL to about 500 mL, or from about 100 mL to about 500 mL, or from about 250 to about 500 mL.

In some aspects, an HSC composition is provided with a hyperresponsive EPOR (as described). The HSC composition of the present disclosure comprises at least 0.0001% LT-HSCs. In some embodiments of this disclosure, the HSCs comprise at least about 0.05% LT-HSCs, or at least about 0.1% LT-HSCs, or at least about 0.5% LT-HSCs, or at least about 1% LT-HSCs. In other aspects, the invention provides a cell population, or pharmaceutically acceptable composition thereof, produced by the method described herein. In various embodiments, the composition for RBC therapy is prepared that comprises the cell population and a pharmaceutically acceptable vehicle. The pharmaceutical composition may comprise at least about 10² cells, or at least about 10³, or at least about 10⁴, or at least about 10⁵, or at least about 10⁶, or at least about 10⁷, or at least about 10⁸ cells, or at least about 10⁹ cells, or at least about 10¹⁰ cells, or at least about 10¹¹ cells, or at least about 10¹² cells, or at least about 10¹³ cells, or at least about 10¹⁴ cells per kilogram body weight. For example, in some embodiments, the pharmaceutical composition is administered, comprising HSCs (e.g., having a hyperresponsive EPOR) in the range of about 100,000 to about 400,000 cells per kilogram (e.g., about 200,000 cells/kg). In other embodiments, RBCs are administered at from about 10⁵ to about 5×10⁵ cells per kilogram (e.g., about 2.5×10⁵ cells/kg), or from about 10⁶ to about 5×10⁶ cells per kilogram (e.g., about 2.55×10⁶ cells/kg), or from about 5×10⁶ to about 10⁷ cells per kilogram (e.g., about 5×10⁶ cells/kg) or from about 10⁷ to about 10⁸ cells per kilogram (e.g., about 5×10⁷ cells/kg) or from about 10⁸ to about 10⁹ cells per kilogram (e.g., about 5×10⁸ cells/kg) or from about 10⁹ to about 10¹⁰ cells per kilogram or from about 10¹⁰ to about 10¹¹ cells or from about 10¹¹ to about 10¹² cells per kilogram or from about 10¹² to about 10¹¹¹ cells per kilogram or from about 10¹³ to about 10¹¹ cells per kilogram of a recipient's body weight.

In some embodiments (as described), the HSCs are HLA-A^{neg}, homozygous for both HLA-B and HLA-C, HLA-DPB1^{neg}, and HLA-DQB1^{neg}. In some embodiments, the iPSCs are further homozygous for HLA-DRB1.

The cell composition of this disclosure may further comprise a pharmaceutically acceptable carrier or vehicle suitable for intravenous infusion or other administration route, and the composition may include a suitable cryoprotectant. An exemplary carrier is DMSO (e.g., about 10% DMSO). Cell compositions may be provided in unit vials or bags and stored frozen until use.

Cells produced according to this disclosure can be administered or used in therapy, for example, for an inherited or acquired red cell disorder, bone marrow failure disorder, high-altitude-related physiological and pathological condition, anemia (e.g., sickle cell anemia), red cell enzyme deficiencies (e.g. G6PD), red cell membrane disorders (e.g. hereditary spherocytosis), hemoglobinopathies (e.g. sickle cell disease and thalassemia), hemolytic anemia, nutritional anemias (e.g. iron deficiency anemia, and folate deficiency), disorders of heme production (e.g. sideroblastic anemia), hemochromatosis, conditions related to chemicals or radiation exposure, and/or for treatment of subjects undergoing HSC transplant. In further embodiments, the red cells prepared according to this disclosure are provided as a pharmaceutical acceptable composition delivering or encapsulating drugs (including but not limited to enzymes), oxygen carriers, or other suitable materials to treat human disease or physiological or pathological conditions.

In certain embodiments, the present disclosure provides compositions and methods for treating anemia. In embodiments, the present disclosure provides for hematopoietic stem cell (HSC) compositions or erythroid progenitor compositions that provide for a durable and potent cell therapy for anemia. In embodiments, the HSCs are supercharged with an erythropoietin receptor (EPOR) that is truncated and/or mutated, resulting in a hyperresponsive EPOR. In embodiments, the compositions and methods of the present disclosure can provide patients with sufficient red blood cells to supply healthy oxygenation levels. In embodiments, the compositions and methods of the present disclosure effectively utilize the potential of induced pluripotent stem cells (iPSCs) to produce HSC populations comprising a significant number of Long-Term (LT)-HSCs for therapy, thereby providing durable production of red blood cells in vivo.

Anemia is a condition where one lacks enough healthy red blood cells to carry adequate oxygen to body tissues. Principal symptoms of anemia include feeling tired and weak. There are many forms of anemia, such as sickle cell anemia, aplastic anemia, and anemia associated with a bone marrow disease or bone marrow failure, among others. For example, sickle cell disease is a group of inherited red blood cell disorders in which the patients have mutations in the beta globin gene that leads to abnormal hemoglobin, called hemoglobin S. Hemoglobin S changes flexible red blood cells into rigid, sickle-shaped cells. Sickle cell anemia is the most common and most severe type of sickle cell disease where the red blood cells die early, leaving a shortage of healthy red blood cells.

Current treatments of sickle cell disease, and other types of anemia include blood transfusions and blood and bone marrow transplant. Patients undergoing available treatments continue to face significant difficulties and complications. For instance, patients treated with long-term blood transfusion therapy, e.g., to increase oxygen-carrying capacity and to decrease the proportion of sickle hemoglobin (HbS) relative to hemoglobin A (HbA), face a significant burden, including the need for regular hospital attendance and are often required to take iron chelation therapy. Howard J. Sickle cell disease: when and how to transfuse. Hematology Am Soc Hematol Educ Program. 2016; 2016(1):625-631. Patients treated with blood and bone marrow transplant, i.e., replacing patients' hematopoietic stem cells with those from donors, continue to face significant risks including graft-versus-host disease, exposure to infection, and the need for chemotherapy. Rangarajan, H. G., Abu-Arja, R., Pai, V., Guilcher, G., & Soni, S. Outcomes of Unrelated Donor Stem Cell Transplantion with Post-Transplant Cyclophosphamide for Graft-versus-Host Disease Prophylaxis in Patients with Severe Sickle Cell Disease. Biology of Blood and Marrow Transplantation. 2018; 24(2), 413-417.

HSCs or erythroid progenitors generated using the methods described herein are administered to a subject (a recipient), e.g., by intravenous infusion or intra-bone marrow transplantation. The methods can be performed following myeloablative, non-myeloablative, or immunotoxin-based (e.g., anti-c-Kit, anti-CD45, etc.) conditioning regimes. In some embodiments, the methods are performed without myeloablative, non-myeloablative, or immunotoxin-based (e.g., anti-c-Kit, anti-CD45, etc.) conditioning regimes.

In various embodiments, the present disclosure provides a method of treating a subject in need of red blood cell production, comprising administering to the subject the HSC or erythroid progenitor composition of the present disclosure. As such, the HSCs produced according to this disclosure may be used to produce red blood cells in vivo in a durable and potent manner.

In some embodiments, the recipient subject has anemia. In some embodiments, the recipient subject has sickle cell anemia, aplastic anemia, anemia associated with a bone marrow disease or bone marrow failure, anemia from blood loss, or hemolytic anemia. In some embodiments, the recipient subject has Fanconi anemia. In some embodiments, the subject has thalassemia. In some embodiments, the HSCs or erythroid progenitors are used to prepare blood products for treating co-morbidities associated with blood, bone marrow, immune, metabolic, or mitochondrial disorders.

As used herein, the term “about” means 10% of the associated numerical value.

Certain aspects and embodiments of this disclosure are further described with reference to the following examples.

### EXAMPLES

### Example 1—ETV2 Over-Expression Increases the Yield of Hemogenic Endothelial Cells and Enhances the CD34+ Cell Formulation During iPSC Differentiation but does not Affect Pluripotency

### Methods

iPSCs were developed from hCD34+ cells by episomal reprogramming as known in the art and essentially as described in Yu, et al. Induced pluripotent stem cell lines derived from human somatic cells, Science 318, 1917-1920, (2007); and J. Yu, et al. Human induced pluripotent stem cells free of vector and transgene sequences. Science 324, 797-801, (2009). Embryoid Bodies and hemogenic endothelium differentiation was performed essentially as described in: R. Sugimura, et al., Haematopoietic stem and progenitor cells from human pluripotent stem cells. Nature 545, 432-438, (2017); C. M. Sturgeon, et al, Wnt signaling controls the specification of definitive and primitive hematopoiesis from human pluripotent stem cells. Nat Biotechnol 32, 554-561, (2014); J. Yu, et al. Induced pluripotent stem cell lines derived from human somatic cells. Science 318, 1917-1920, (2007); and J. Yu, et al. Human induced pluripotent stem cells free of vector and transgene sequences. Science 324, 797-801, (2009).

Briefly, hiPSC were dissociated and resuspended in media supplemented with L-glutamine, penicillin/streptomycin, ascorbic acid, human holo-Transferrin, monothioglycerol, BMP4, and Y-27632. Next, cells were seeded in 10 cm dishes (EZSPHERE or low attachment plate) for the EB formation. On Day 1, bFGF and BMP4 were added to the medium. On Day 2, the media was replaced with a media containing SB431542, CHIR99021, bFGF, and BMP4. On Day 4, the cell media was replaced with a media supplemented with VEGF and bFGF. On day 6, the cell media was replaced with a media supplemented with bFGF, VEGF, interleukin (IL)-6, IGF-1, IL-11, SCF, and EPO. Cells were maintained in a 5% CO2, 5% O2, and 95% humidity incubator. To harvest the CD34+ cells, the EBs were dissociated on day 8, cells were filtered through a 70 μm strainer, and CD34+ cells were isolated by CD34 magnetic bead staining.

### Results

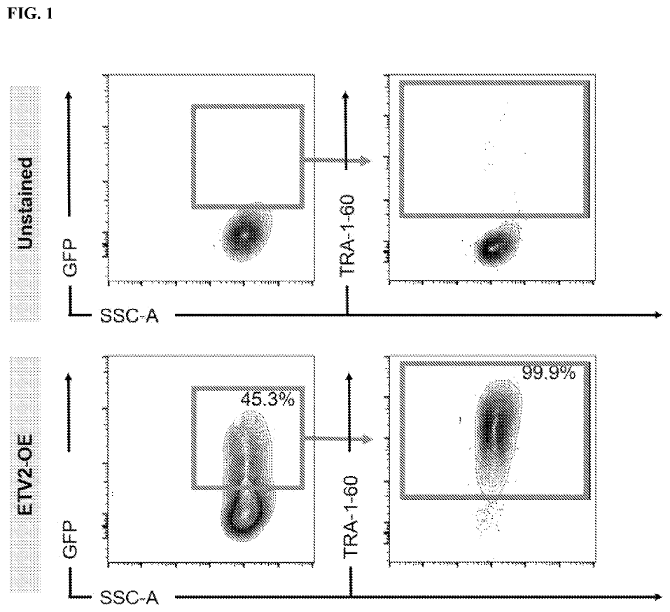

An adenoviral vector containing both ETV2 and GFP sequences under the control of the EF1A promoter was used to transduce induced pluripotent stem cells (iPSCs). After the transduction, about 45% of the iPSC culture was observed to be GFP positive, thus confirming ETV2 overexpression (ETV2-OE). It was further observed that ETV2-OE in iPSC cells preserves the pluripotency properties of iPSCs as shown by the sternness marker expression TRA-1-60 (FIG. 1). FIG. 1 shows FACS plots representative of transduction efficiency of iPSC with an adenoviral vector to overexpress the ETV2 and the GFP sequences.

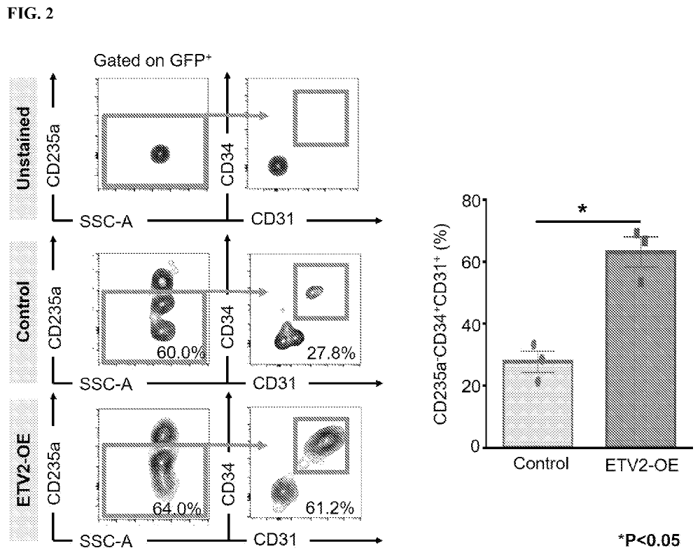

Next, the ETV2-OE-iPSCs were differentiated (along with control iPSCs transduced with a vector bearing the GFP sequence without ETV2) to embryoid bodies and subsequently to hemogenic endothelial cells (Strugeon et al., 2014). The results suggest that the overexpression of ETV2 boosts the formation of hemogenic endothelial cells as demonstrated by the expression of the CD34+ and CD31+ markers within the CD235a-population (FIG. 2). Specifically, FIG. 2 shows representative flow cytometric analysis of hemogenic endothelial cells (defined here as CD235a-CD34+CD31+) and relative quantification demonstrates that ETV2-OE enhances the formation of hemogenic endothelial cells as compared to controls.

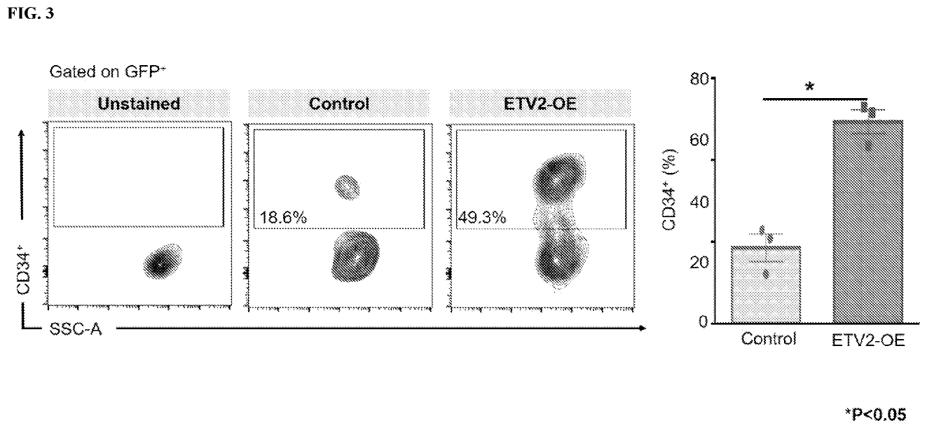

Moreover, the results suggest that ETV2-OE enhances the formation of the CD34+ cells (FIG. 3). FIG. 3 shows representative flow cytometric analysis of CD34+ cells and relative quantification demonstrates that ETV2-OE enhances the CD34+ cell formation.

Overall, these data indicate that ETV2 overexpression in iPSCs does not affect their pluripotency properties and facilitates their ability to undergo the hemogenic endothelial and hematopoietic differentiations.

### Example 2—iPSC-Derived HSCs Generated with Piezol Activation Undergo T Cell Differentiation Similar to Bone Marrow-Derived HSCs

### Methods

To analyze the EHT, EB-derived CD34+ cells were suspended in medium containing Y-27632, TPO, IL-3, SCF, IL-6, IL-11, IGF-1, VEGF, bFGF, BMP4, and FLT3. After the cells had adhered to the bottom of the wells for approximately 4-18 hours (by visual inspection), Yodal was added to the cultures. After 4-7 days, the cells were collected for analysis.

iPSCs were differentiated to embryoid bodies for 8 days. At day 8, CD34+ cells from iPSC-derived embryoid bodies were harvested and cultured for additional 5 to 7 days to induce endothelial-to-hematopoietic (EHT) transition. Then, CD34+ cells were harvested from the EHT culture between day 5 to day 7 for further hematopoietic lineage differentiation.

CD34+ cells, harvested from the EHT culture between day 5-7 (or total of day 13-21 differentiation from iPSCs), were seeded in 48-well plates pre-coated with rhDL4 and RetroNectin. T lineage differentiation was induced in media containing aMEM, FBS, ITS-G, 2BME, ascorbic acid-2-phosphate, Glutamax, rhSCF, rhTPO, rhIL7, FLT3L, rhSDF-1a, and SB203580.

Between day 2 to day 6, 80% of the media was changed every other day. At D7, cells were transferred into new coated plates and analyzed for the presence of pro-T cells (CD34+CD7+CD5+/−).

Between day 8 to day 13, 80% of the media was changed every other day. At D14, 100,000 cells/wells were transferred to a new coated plate and the cells analyzed for the presence of pre-T cells (CD34− CD7+ CD5+/−).

Between day 15 to day 20, 80% of the media was changed every other day. Cells were harvested at D21, and the cells were analyzed for CD3, CD8, CD5, CD7, TCRab expression, as surrogates for T cells, via FACS, and/or activated using CD3/CD28 beads to evaluate their functional properties.

After 21 days of differentiation, cells were collected and re-seeded at approximately 80,000 cells into new 96-well culture plates in RPMI 1640 (no L-glutamine; no phenol red) plus FBS, L-glutamine, IL-2, and then activated with 1:1 CD3/CD28 beads. After 72 hours of activation with CD3/CD28 beads, cells were analyzed for CD3, CD69, CD25 expression by FACS and IFN-γ expression using RT-qPCR. The supernatant was analyzed by ELISA.

### Results

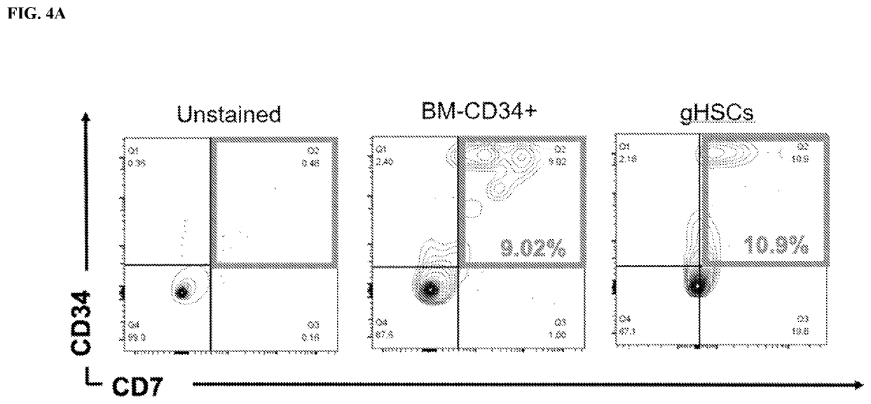

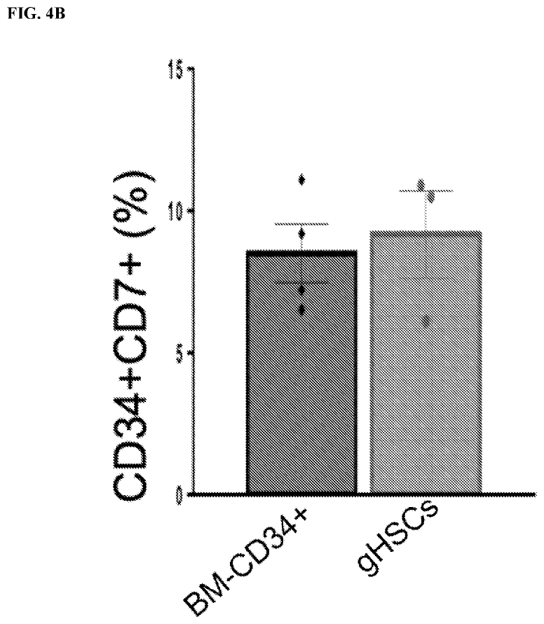

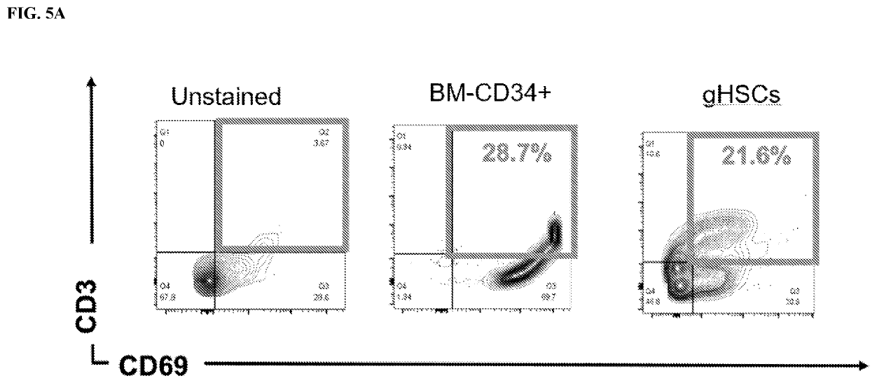

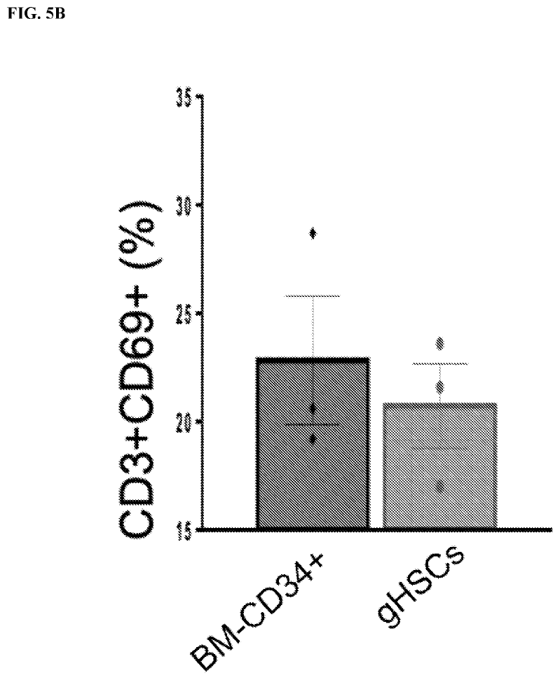

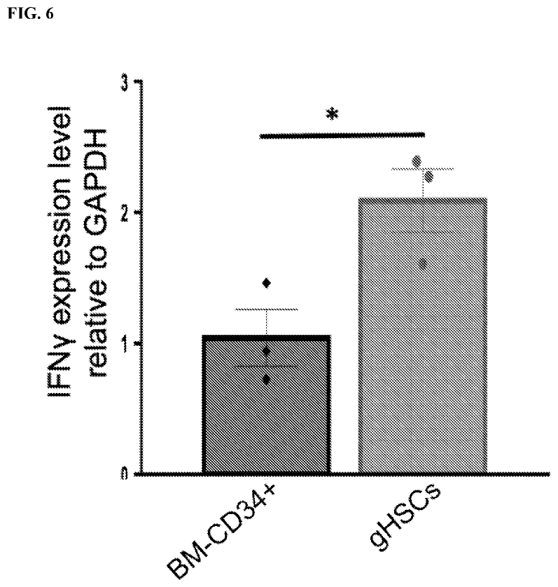

FIG. 4A and FIG. 4B show that iPSC-derived HSCs that are derived with EHT of Day8 34+ cells (in this example with Piezol activation) undergo pro-T cell differentiation similar to bone marrow (BM)-HSCs. Further, FIG. 5A and FIB. 5B show that iPSC-derived HSCs generated with EHT of D8 34+ cells (in this case with Piezol activation) undergo T cell differentiation and can be activated with CD3/CD28 beads similar to BM-HSCs. FIG. 6 shows that iPSC-derived HSCs generated with EHT of D8 34+ cells (in this case Piezol activation) can differentiate to functional T cells, as demonstrated by INFγ expression upon stimulation with CD3/CD28 beads. Together, these results demonstrate that EHT of 34+ cells from differentiated iPSCs enhances HSC ability to further differentiate to hematopoietic lineages, such as progenitor T cells and functional T cells ex vivo, among others.

### Example 3 Evaluating Off-Target Editing in HLA Knockout HSCs

HLA typing of the triple knockout (HLA edited) HSC clones was performed to check for unwanted editing and to ensure that no major editing events, e.g., deletion(s), occurred within other regions of chromosome 6. Sequencing methods and analyses were performed to evaluate the degree of gRNA off-target activity and to select gRNAs that represent a low risk of affecting non-target HLA genes.

Sequencing was performed by using in situ break labelling in fixed and permeabilized cells by ligating a full-length P5 sequencing adapter to end-prepared DSBs. Genomic DNA was extracted, fragmented, end-prepared, and ligated using a chemically modified half-functional P7 adapter. The resulting DNA libraries contained a mixture of functional DSB-labelled fragments (P5:P7) and non-functional genomic DNA fragments (P7:P7). Subsequent DNA sequencing of the DNA libraries enriched for DNA-labelled fragments, eliminating all extraneous, non-functional DNA. As the library preparation is PCR-free, each sequencing read obtained was equivalent to a single labelled DSB-end from a cell. This generated a DNA break readout, enabling the direct detection and quantification of genomic DSBs by sequencing without the need for error-correction and enabled mapping a clear list of off-target mutations.

Table 1 below summarizes the results of the editing strategy in two representative clones relative to a wild-type cell.

TABLE 1
Clonal HSC HLA knockouts.

| Sample ID | Locus | Allele 1 | Allele 2 | Comments |
|---|---|---|---|---|
| WT (gHSC) | A | A*01:01:01 | A*01:01:01 | Not affected |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | DPB1*01:01:01 | DPB1*04:01:01 | Not affected |
| | DQA1 | DQA1*05:01:01 | DQA1*05:01:01 | Not affected |
| | DQB1 | DQB1*02:01:01 | DQB1*02:01:01 | Not affected |
| | DRB1 | DRB1*03:01:01 | DRB1*03:01:01 | Not affected |
| | DRB345 | DRB3*01:01:02 | DRB3*01:01:02 | Not affected |
| Clone #1 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Delction in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #2 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |

Table 2 provides a non-limiting example of gRNAs used in the experiments which can be used to knock out expression of indicated HLA genes.

TABLE 2
Exemplary gRNA sequences

| gRNA ID | Spacer sequence |
|---|---|
| HLA-A | CGTAGCCCACGGCGATGAAG (SEQ ID NO: 1) |
| | ATTTCTTCACATCCGTGTCC (SEQ ID NO: 2) |
| | GTCTCCTGGTCCCAATACTC (SEQ ID NO: 3) |
| | GTAGCCCACGGCGATGAAGC (SEQ ID NO: 4) |
| | TAGCCCACGGCGATGAAGCG (SEQ ID NO: 5) |
| | GAGGGTTCGGGGCGCCATGA (SEQ ID NO: 6) |
| | CCGGAACACACGGAATGTGA (SEQ ID NO: 7) |
| | ACAGCGACGCCGCGAGCCAG (SEQ ID NO: 8) |
| | CCACTCACAGATTGACCGAG (SEQ ID NO: 9) |
| | CGACGCCGCGAGCCAGAGGA (SEQ ID NO: 10) |
| | CAGATTGACCGAGTGGACCT (SEQ ID NO: 11) |
| | CAGACTGACCGAGTGGACCT (SEQ ID NO: 12) |
| | ACAGACTGACCGAGTGGACC (SEQ ID NO: 13) |
| | GCCGGGCCGGGACACGGATG (SEQ ID NO: 14) |
| | CCAGGAGACACGGAATGTGA (SEQ ID NO: 15) |
| | CCAGTCACAGACTGACCGAG (SEQ ID NO: 16) |
| | TCGACAGCGACGCCGCGAGC (SEQ ID NO: 17) |
| HLA- | GAGATACATCTACAACCGGG (SEQ ID NO: 18) |
| DPB1 | TGGAGAGATACATCTACAAC (SEQ ID NO: 19) |
| | GGTTGTAGATGTATCTCTCC (SEQ ID NO: 20) |
| | GGAGAGATACATCTACAACC (SEQ ID NO: 21) |
| | AGGAATGCTACGCGTTTAAT (SEQ ID NO: 22) |
| HLA- | CAGATACATCTATAACCGAG (SEQ ID NO: 23) |
| DQB1 | CGAGTACTGGAACAGCCAGA (SEQ ID NO: 24) |
| | CCCGTTGGTGAAGTAGCACA (SEQ ID NO: 25) |
| | GTGCTACTTCACCAACGGGA (SEQ ID NO: 26) |
| | AGGTCGTGCGGAGCTCCAAC (SEQ ID NO: 27) |
| | AGGTGACAGTTGCTACCCGA (SEQ ID NO: 28) |
| | TCCCGTTGGTGAAGTAGCAC (SEQ ID NO: 29) |
| | AACTACGAGGTGGCGTACCG (SEQ ID NO: 30) |
| | CTGTTCCAGTACTCGGCAAC (SEQ ID NO: 31) |
| | ACTACGAGGTGGCGTACCGC (SEQ ID NO: 32) |
| | CGAAGCGCACGTACTCCTCT (SEQ ID NO: 33) |

The results show that the editing strategy was successful in selectively targeting the HLA-A, DPB1, and DQB1 genes without affecting the other HLA genes or introducing major deletion elsewhere.

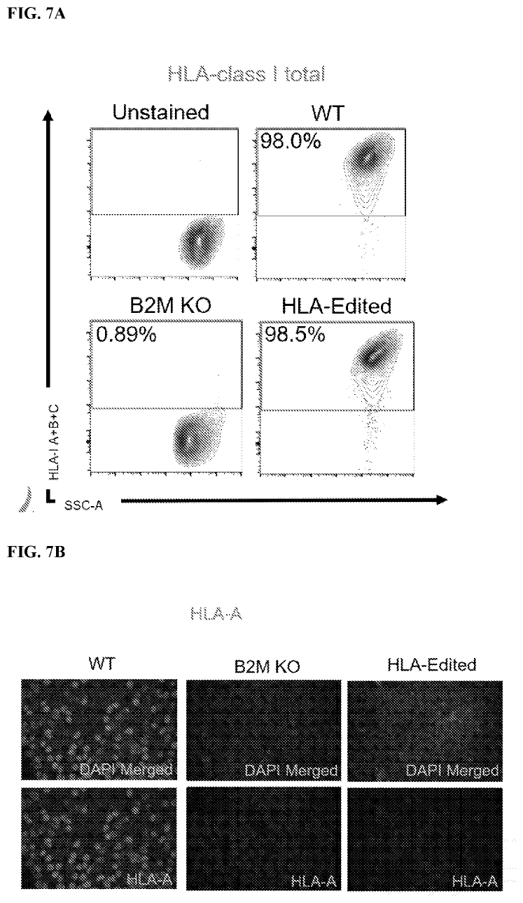

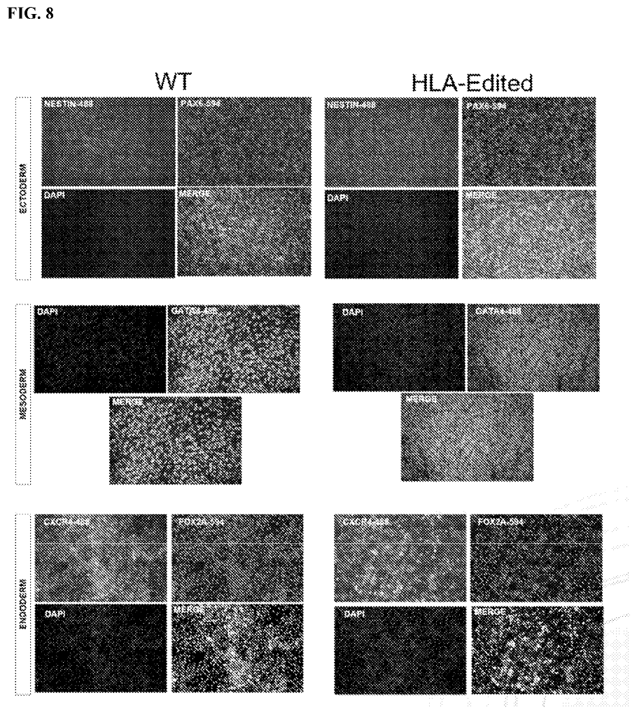

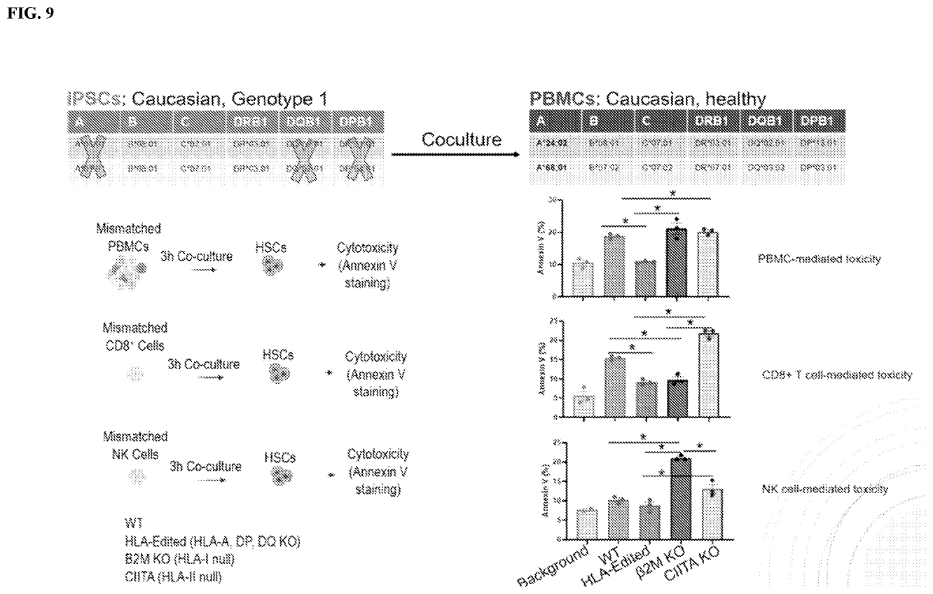

These results were confirmed by a phenotypic analysis of the HILA edited clones by FACS and immunofluorescence. As shown in FIGS. 9A and B, HLA edited cells tested positive for overall expression of HLA class-I molecules, comparable to the overall expression of HLA class-I molecules of wild-type cells. Specific expression of HLA-A via immunofluorescence confirmed that HLA-A was not expressed in the HLA edited cells, corroborating the finding that the gene editing strategy was successful in deleting only the HLA-A gene. Specifically, FIG. 7A, shows that the HLA edited cells were all positive for class-I like HLA to the same extent as the wild type (WT) (i.e., non-HLA-edited) cells. This result indicates that despite the deletion of HLA-A, other class-I molecules like HLA-B and C were expressed and not affected by the gene editing strategy.

To confirm that the HLA-A gene was deleted, specific expression of the HLA-A was analyzed with immunofluorescence. As can be seen in FIG. 7B, HLA-A was not expressed in the HLA edited clone indicating that the gene editing strategy was efficient in specifically deleting the HLA-A gene only. Such preservation of overall class-I expression with deletion of HLA-A will facilitate patient matching while avoiding NK-cell mediated rejection.

### Example 4 Evaluating Pluripotency and Immunocompatibility of HLA Edited HSCs

The ability of HLA edited cells to preserve pluripotency was evaluated. As shown in FIG. 8, immunofluorescence evaluation of the HLA edited iPSC clones indicated that they maintained trilineage differentiation, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

HLA class I molecules are expressed on the surface of all nucleated cells and if the HLA class I molecules are mismatched between donor and recipient, then the cells could be recognized and killed by CD8+ T cells. Additionally, HLA mismatching could lead to cytokine release syndrome (CRS) and graft-versus-host disease (GVD). Conversely, the complete deletion of HLA-I molecules, via B2M KO, would make the cell a target of NK cell-mediated cytotoxicity. The preservation of overall class-I expression with deletion of HLA-A can facilitate patient matching while preventing the NK-cell mediated rejection. Thus, the immunocompatibility of the HLA edited HSCs was tested by co-culture with peripheral blood mononuclear cells (PBMCs) to evaluate if the immune cells would reject a graft of the HSCs.

Wild-type (WT) and HLA edited HSCs were co-cultured with PBMCs matching the HLA-B and HLA-C markers, but with mismatched HLA-A. B2M KO HSCs lacking expression of HLA class-I molecules and CIITA KO HSCs lacking expression of class-II molecules were used as controls to compare the degree of PBMC-mediated cytotoxicity for HLA-null and mismatched HLA, respectively. FIG. 9 shows the results of the PBMC-mediated cytotoxicity assay in the co-cultures as measured by an annexin V staining. The results show that deletion of HLA-A in the HLA edited HSCs protects the cells from PBMC-mediated cytotoxicity, while WT, B2M KO, and CIITA KO were susceptible to PBMC-mediated cytotoxicity. Co-cultured HSCs with sorted CD8+ T cells from the same PBMC donor protected HLA edited and B2M KO HSCs from CD8+ T cell cytotoxicity. Conversely, co-cultured HSCs with sorted NK cells only protected the WT and HLA edited cells from the NK cell-mediated cytotoxicity.

In summary, the immune compatibility results show that the CD8+ T cells present in the PBMC samples were responsible for killing the cells with mismatched HLA molecules (WT and CIITA KO), while the NK cells present in the PBMCs were responsible for killing the HLA-null cells (B2M KO). However, HLA edited HSCs are protected from CD8+ T cell-mediated cytotoxicity (because the mismatched HLA-A had been knocked out), and protected from NK cell-mediated cytotoxicity (because HLA class I molecule expression was largely preserved).

### Example 5 Evaluating the In Vivo Engraftment Potential of HLA Edited HSCs

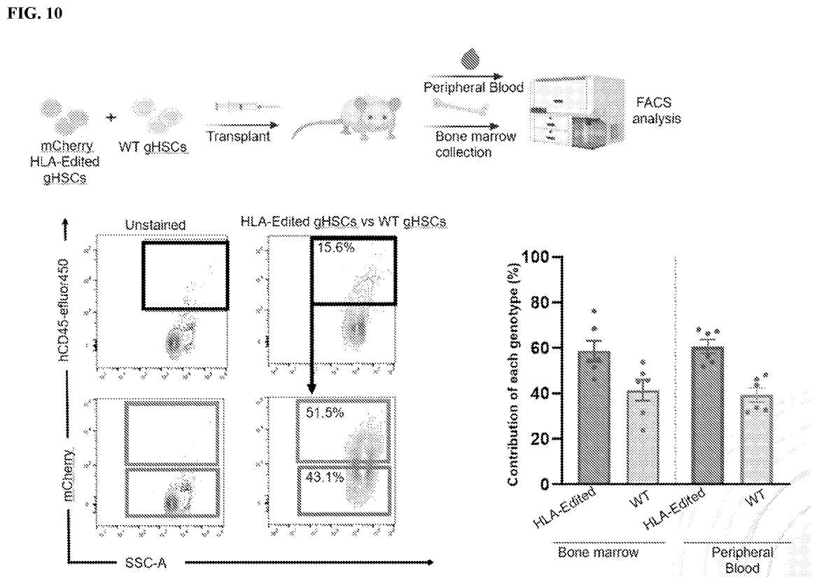

To evaluate the engrafting potential of HLA edited HSCs, the cells' ability to engraft in vivo was evaluated by a competitive transplant against WT HSCs. Equal proportions of mCherry HLA edited HSCs and wild-type HSCs were admixed and transplanted into mice, where bone marrow (BM) and peripheral blood samples were recovered and evaluated by FACS to compare the relative amounts of each cell type present in the samples. As shown in FIG. 10, both the HLA edited HSCs and the WT HSCs contributed to approximately equal engraftment in the BM and peripheral blood samples. These results confirm that HLA edited HSCs (prepared according to this disclosure) are comparable to WT HSCs in their engraftment and reconstitution potential. Hence, it is expected that properties of the WT (unedited, parent) HSCs are consistent with that of the HLA edited HSCs of the present disclosure, for generating T cell lineages.

### Example 6 Differentiation of HLA Edited HSCs to CD4+, CD8+ T Cells

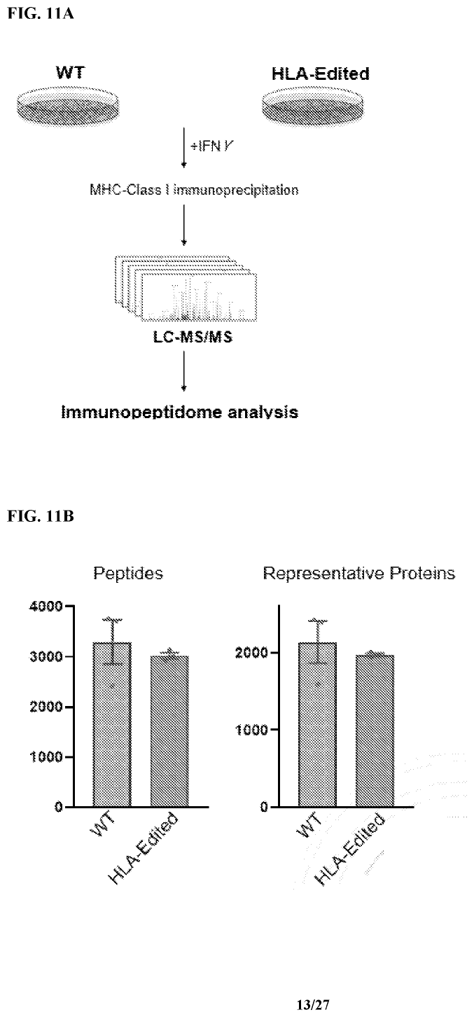

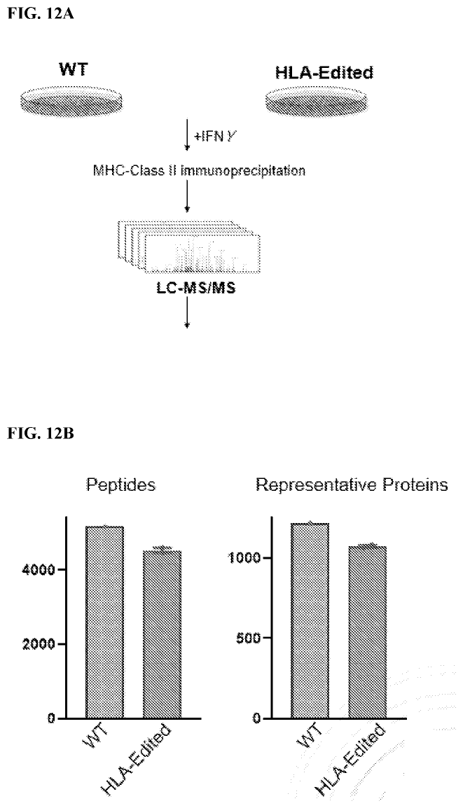

Antigen presenting cells (APCs) present antigens to helper CD4+ T cells through the HLA-II molecules. Activation of helper CD4+ T cells promotes the generation of antigen-specific CD8+ T cells which further develop into antigen-specific CTLs. Likewise, HLA Class I molecules are expressed on the surface of all nucleated cells and display peptide fragments of proteins from within the cell to CD8+ CTLs. CTLs induce cytotoxic killing of target (infected) cells upon recognition of HLA-I−peptide complex expressed on the cell surface. Hence, a study was carried out to determine if deletion of HLA-A impacts the edited HSCs' class I peptide presentation. As shown in FIGS. 11A and B, immunopeptidome analysis shows that the deletion of HLA-A does not impact overall class I peptide presentation. HLA-A edited cells showed comparable peptide and protein presentation when compared to wild type (non-HLA-edited) HSCs. Further, as shown in FIGS. 12A and B, deletion of HLA-DQB1 and HLA-DPB1 does not impact overall class II peptide presentation by macrophages differentiated from the HSCs. Together, these data suggest that despite the deletion of HLA-A, HLA-DQ, and HLA-DP molecules, the cells (and their derived lineages) preserve their ability to present a broad spectrum of class I and II peptides.

### Example 7 In Vivo Testing of Antigen-Mediated Immune Response

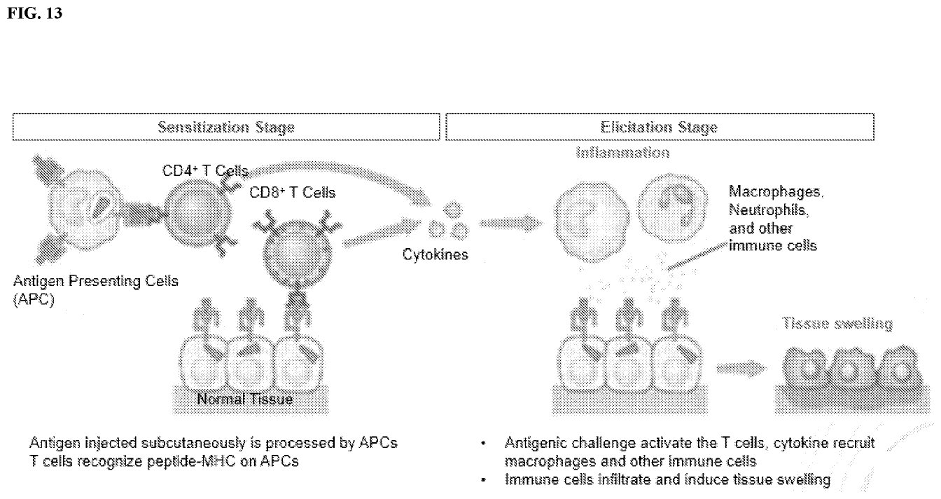

FIG. 13 is a schematic illustration of a Delayed Type Hypersensitivity Reaction, showing the sensitizing and eliciting stages of an antigen presentation. Briefly, upon antigen injection, antigen is processed by antigen presenting cells (APC) and presented by MHC Class II molecules on the APC surface. CD4+ T cells recognize peptide-MHC on antigen presenting cells (APCs). Upon antigenic challenge CD4+ helper T cells are activated and cytokines recruit macrophages and other immune cells, which induce tissue swelling.

A delayed-type hypersensitivity assay was performed on transplanted mice. Specifically, the mice were sensitized by subcutaneous injection of sheep Red blood cells as antigen. If the mice have a functional immune system, the APCs process the antigen and present peptide antigens to CD4+ T cells. Next, the mice were challenged by subcutaneous injection of the same antigen in the left paw. At this point the T cells are activated and secrete cytokines which recruit macrophages and other immune cells at the site of antigen injection creating tissue swelling. In this assay, a functional immune system resulted in the swelling of the left paw as measured with a micro caliper.

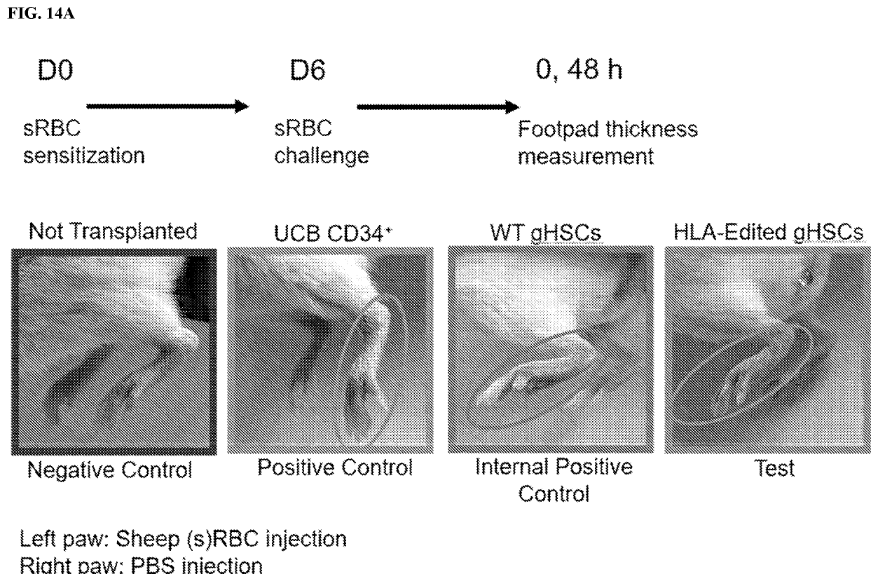

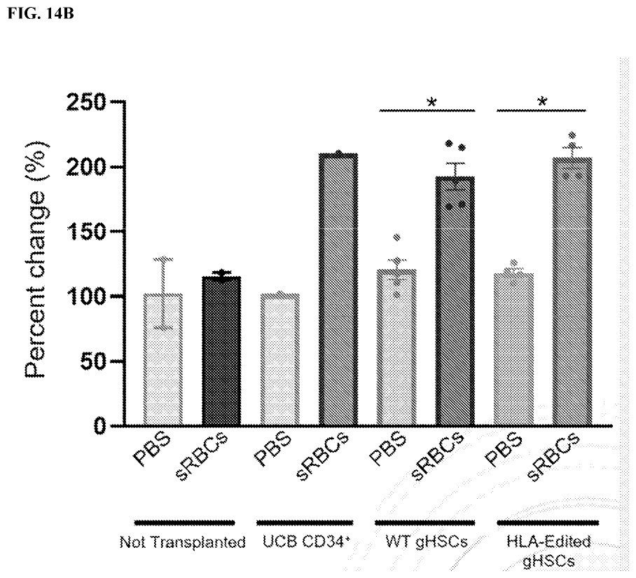

As can be seen in FIGS. 14A and B, the control (non-transplanted) mice did not show any left paw swelling as they are immunodeficient. Conversely, the mice transplanted with Cord Blood CD34+ cells showed tissue swelling and doubled the diameter of their left paw. A similar immune system response was found in both the mice transplanted with the WT (non-edited HSCs) and the HLA-edited HSCs.

### Example 8—Evaluating HSC-Derived T Cell (Pro-T Cell) Differentiation and Maturation

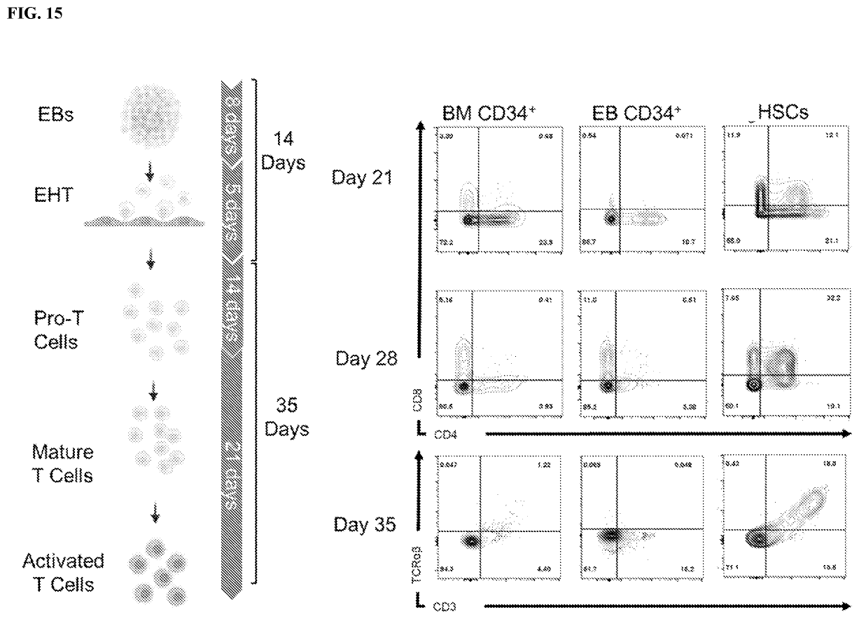

Next, the ability of the HSC-derived T cells (pro-T cells) to differentiate into mature T cells was tested. After a 35-day differentiation period, pro-T cells were evaluated by cell sorting for the presence of CD4+, CD8+, and AB+ T cell populations. As shown in FIG. 15, pro-T cells differentiated into CD4+, CD8+, and αβ+ T cells more efficiently than bone marrow (BM)-derived CD34+ cells and the CD34+ cells derived from the embryonic bodies (EB).

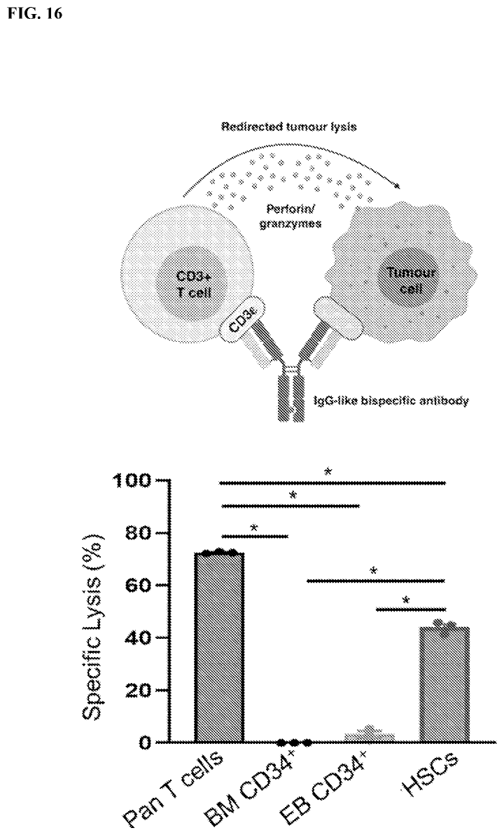

Next, to test the functional properties, each of the T cell populations were co-cultured with a CD19+ lymphoma cell line and an anti-CD3/CD-19 bispecific antibody. In this experimental model, the bispecific antibody engaged both the CD3 receptor on T cells and the CD19 cell surface receptor of the lymphoma cells, thus triggering T cell activation. The degree of activation was evaluated by measuring the subsequent T-cell mediated cytotoxicity in comparison to a Pan T cell positive control. As shown in FIG. 16, the pro-T cells exhibited a statistically significant outperformance in cytotoxicity in comparison to both the BM CD34+ T cells and the EB CD34+ T cells.

### Example 9—Evaluating HSC Properties of Developing into Pro-T Cells

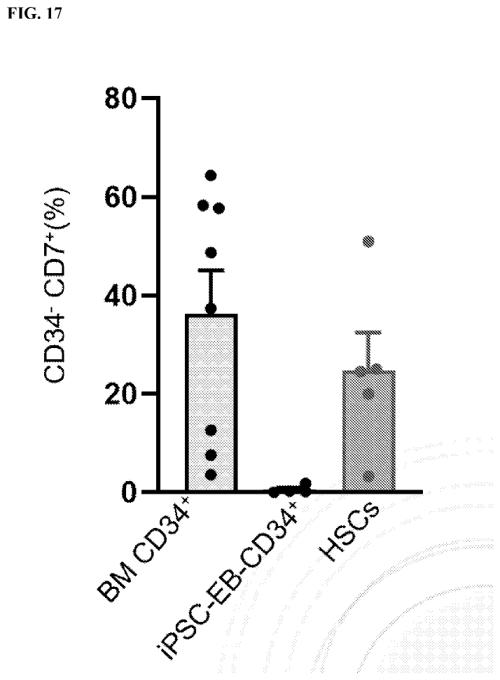

The ability of the HSCs to develop into pro-T cells was assessed by measuring the CD34−CD7+ markers on the pro-T cells. As shown in FIG. 17, FACS analysis showed that HSCs produced according to this disclosure successfully differentiated into CD34−CD7+ pro-T cells, as compared to bone marrow derived CD34+ cells or EB-derived CD34+ cells.

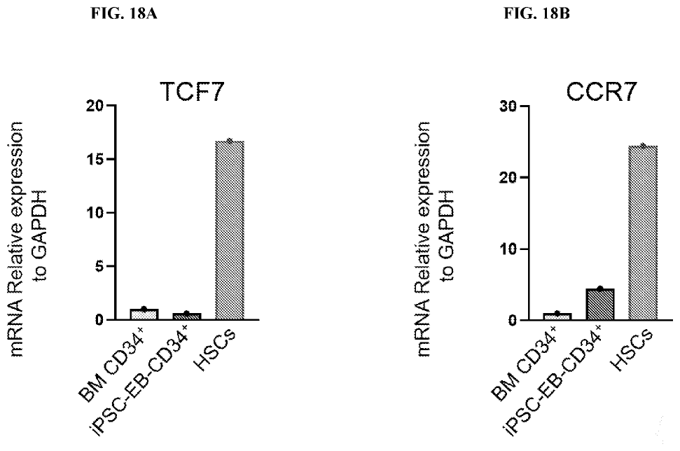

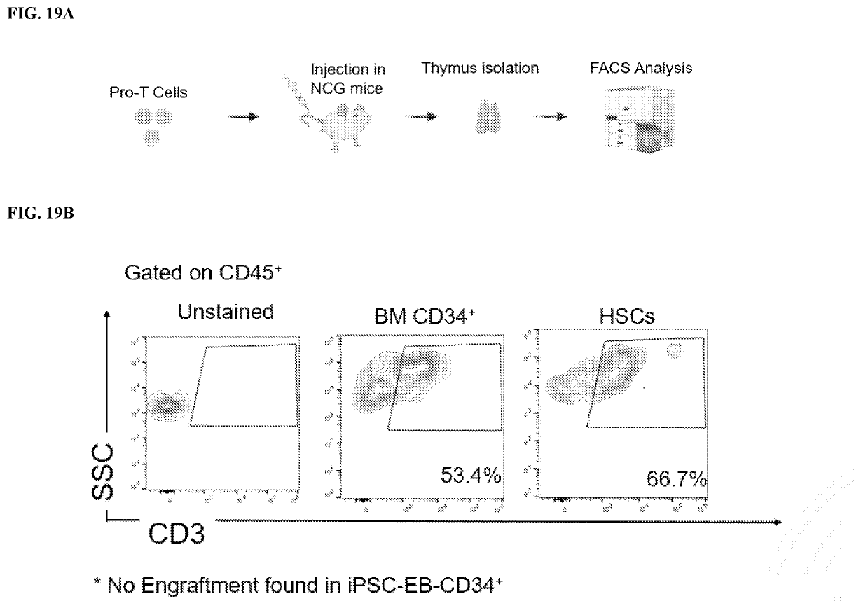

Next, the expression of T cell-specific transcription factors and thymus engrafting molecules were measured. FIG. 18A shows increased TCF7 expression and FIG. 18B shows increased CCR7 expression in the HSC-derived pro-T cells of the disclosure. FIG. 19A shows HSCs-derived Pro-T Cells engraft and differentiate in thymus. FIG. 19B shows FACS analysis of CD3 cell population of cells gated on a CD45+ cell population, which shows the superior engraftment and differentiation potential of the HSCs-derived Pro-T Cells in the thymus. Pro-T Cells of this example were prepared from HSCs using Piezol activation as already described.

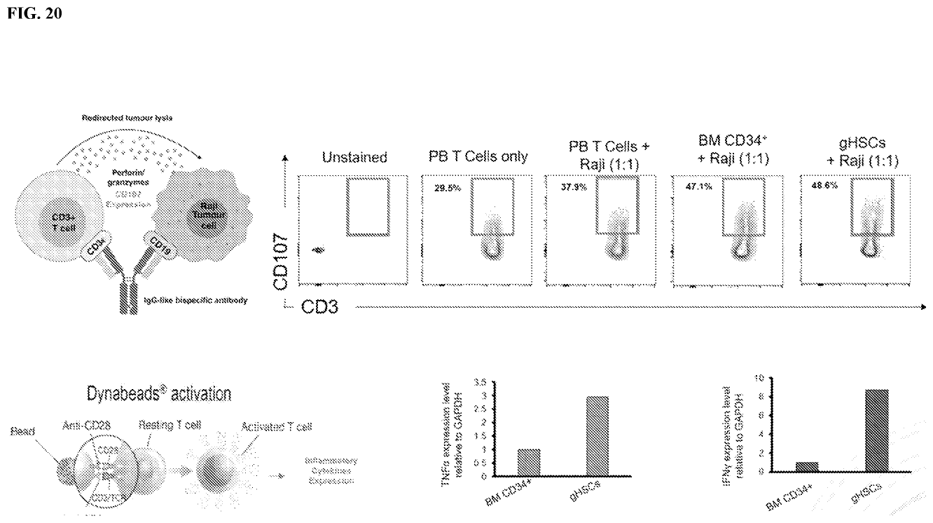

An in-vitro activation of the HSC-derived T cells were also measured, as illustrated in FIG. 20. Top panel of FIG. 20 shows FACS analysis of activated T cells from different sources, including the HSCs of the present disclosure (e.g., prepared using Piezol activation). T cells prepared from HSCs of the present disclosure demonstrated comparable or superior activation as measured by increased CD107 expression. The lower panel shows Dynabeads activation, where activated T cells express inflammatory cytokines. HSC-derived T cells according to the present disclosure (e.g., prepared using Piezol activation) expressed higher levels of inflammatory cytokines as exemplified by TNF-alpha and interferon gamma expression levels.

### Example 10: Differentiation of HLA Edited HSCs to Hematopoietic Lineages, Pro-Monocyte Macrophage Cells

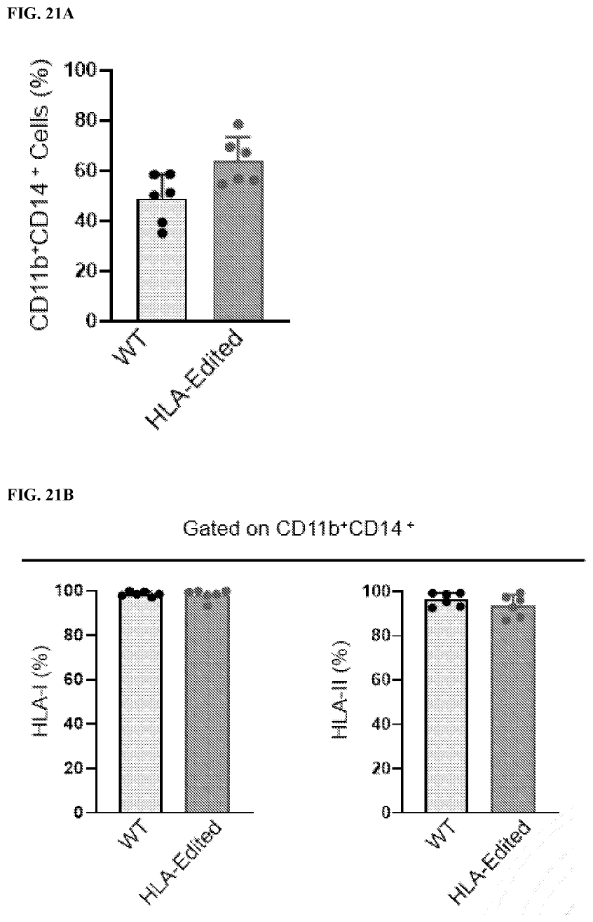

Experiments were carried out to determine if HLA deletion impacts the HSCs ability to differentiate into different types of immune cells. Using the process essentially as described in Example 2, HLA edited HSCs were differentiated to pro-Monocyte/Macrophage cells. It was determined that the HLA-edited HSCs were able to differentiate into monocyte/macrophage lineage comparable to WT (non-HLA-edited) HSCs as measured by their CD11b+−CD14+ expressions (FIG. 21A). Further, the CD11b+−CD14+ gated population showed equivalent HLA-I and HLA-II expression (FIG. 21B) indicating that HLA-edited HSCs also preserve the overall expression of both class I and class II molecules.

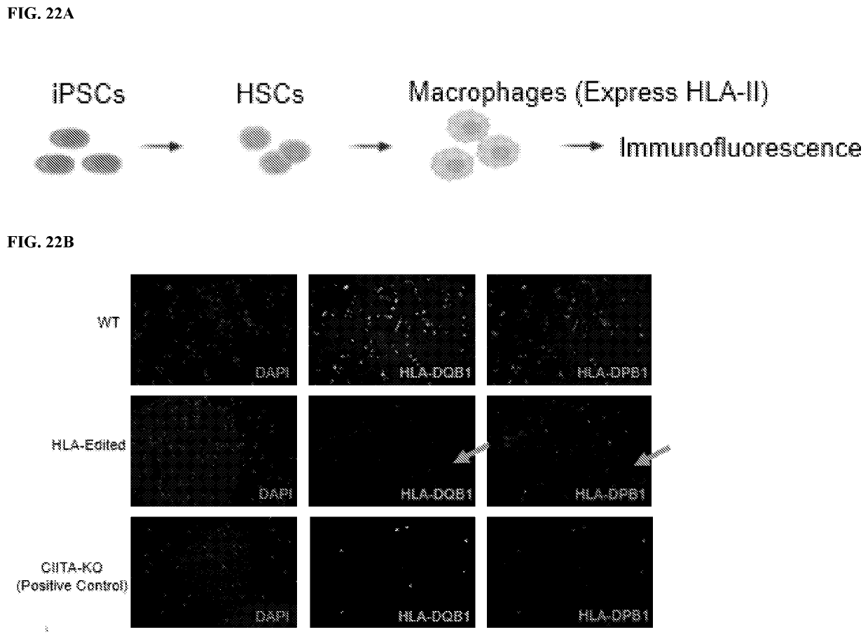

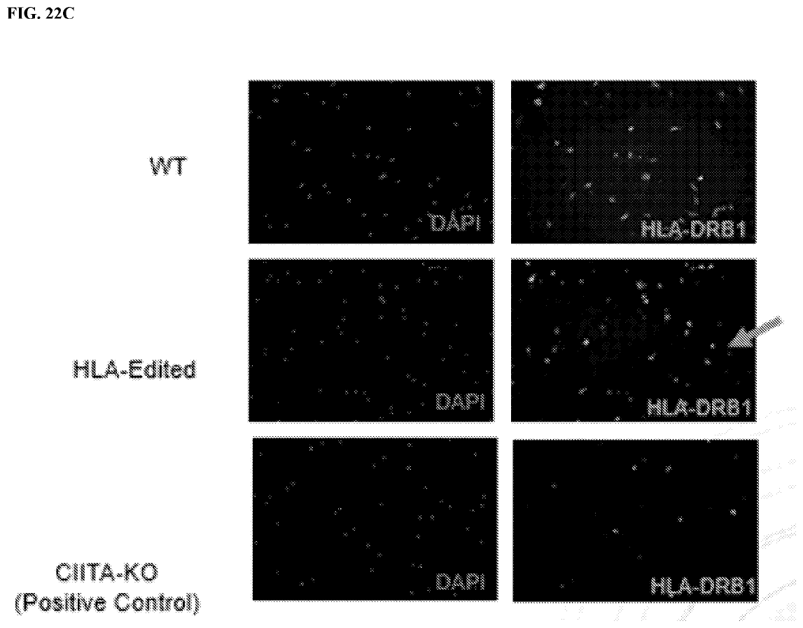

The overall expression of the other class-II molecules in HLA-DQB1 and HLA-DPB1 supported by the edited HSCs was evaluated, by evaluating the expression in macrophages differentiated from the HSCs. The design of the study is schematically shown in FIG. 22A. It was found that the deletion of HLA-DQB1 and HLA-DPB1 did not affect the expression of other HLA Class II molecules (FIG. 22B). For example, HLA-DR is comparably expressed in both WT and HLA-edited cells (FIG. 22C). In FIGS. 22B and 22C, CIITA-KO is as a positive control.

### Example 11: Differentiation of HLA Edited HSCs to Pro-Platelets

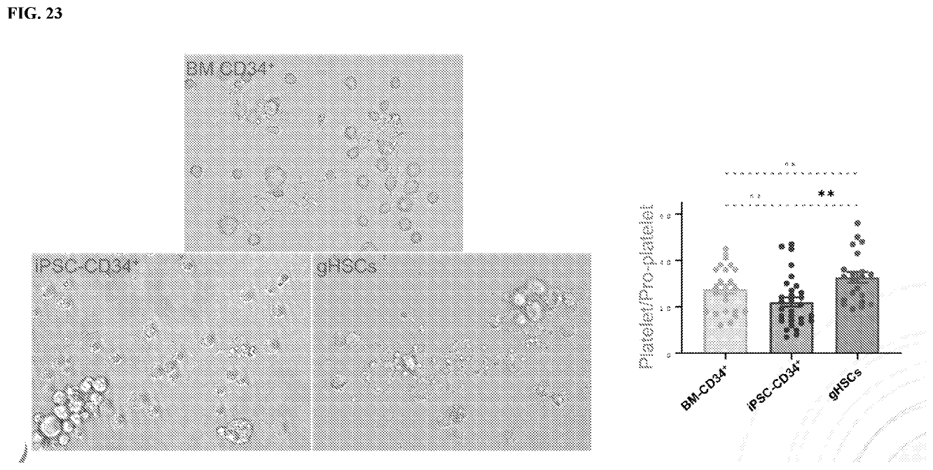

It was determined that the HLA-edited HSCs were able to differentiate into megakaryocytes (MK) and further into platelets. Differentiation was compared against bone marrow (BM)-derived CD34+ cells and iPSC-CD34+ cells. As shown in FIG. 23, the HLA-edited HSCs exhibited a statistically significant increase in platelet content compared to the BM CD34+ and iPSC-34+ cell populations. Thus, HLA-edited HSCs can differentiate into megakaryocytes (MK) which can further support differentiation into platelets.

### REFERENCES

1. Nianias, A. & Themeli, M. Induced Pluripotent Stem Cell (iPSC)-Derived Lymphocytes for Adoptive Cell Immunotherapy: Recent Advances and Challenges. Curr Hematol Malig Rep 14, 261-268 (2019).
2. Brauer, P. M., Singh, J., Xhiku, S. & Znniga-PflIcker, J. C. T Cell Genesis: In Vitro Veritas Est?Trends Immunol 37, 889-901 (2016).
3. Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012).
4. Sturgeon, C. M., Ditadi, A., Awong, G., Kennedy, M. & Keller, G. Wnt Signaling Controls the Specification of Definitive and Primitive Hematopoiesis From Human Pluripotent Stem Cells. Nat Biotechnol 32, 554-561 (2014).
5. Chang, C.-W., Lai, Y.-S., Lamb, L. S. & Townes, T. M. Broad T-Cell Receptor Repertoire in T-Lymphocytes Derived from Human Induced Pluripotent Stem Cells. PLoS One 9, (2014).
6. Nishimura, T. et al. Generation of Rejuvenated Antigen-Specific T Cells by Reprogramming to Pluripotency and Redifferentiation. Cell Stem Cell 12, 114-126 (2013).
7. Themeli, M. et al. Generation of tumor-targeted human T lymphocytes from induced pluripotent stem cells for cancer therapy. Nat Biotechnol 31, 928-933 (2013).
8. Vizcardo, R. et al. Regeneration of Human Tumor Antigen-Specific T Cells from iPSCs Derived from Mature CD8+ T Cells. Cell Stem Cell 12, 31-36 (2013).
9. Montel-Hagen, A. et al. Organoid-induced differentiation of conventional T cells from human pluripotent stem cells. Cell Stem Cell 24, 376-389.e8 (2019).
10. Guo, R. et al. Guiding T lymphopoiesis from pluripotent stem cells by defined transcription factors. Cell Research 30, 21-33 (2020).
11. Nagano, S. et al. High Frequency Production of T Cell-Derived iPSC Clones Capable of Generating Potent Cytotoxic T Cells. Molecular Therapy—Methods & Clinical Development 16, 126-135 (2020).
12. Iriguchi, S. et al. A clinically applicable and scalable method to regenerate T-cells from iPSCs for off-the-shelf T-cell immunotherapy. Nature Communications 12, 430 (2021).

1. A method for preparing a cell population of an erythroid lineage, the method comprising:
enriching for CD34+ cells from a differentiated pluripotent stem cell (PSC) population to prepare a CD34+-enriched population;
inducing endothelial-to-hematopoietic transition of the CD34+-enriched cell population for at least two days, but no more than 12 days, to prepare a population comprising hematopoietic stems cells (HSCs) and/or hematopoietic stem progenitor cells (HSPCs); and
differentiating the population comprising HSCs and HSPCs to a population of an erythroid lineage.

2. The method of claim 1, wherein the PSC population is a human iPSC population derived from erythroblasts, lymphocytes, cord blood cells, peripheral blood mononuclear cells, CD34+ cells, or human primary tissues.

3. (canceled)

4. The method of claim 2, wherein the iPSCs are homozygous for one or more HLA Class I and/er Class II genes.

5. The method of claim 4, wherein the iPSCs are homozygous for HLA-DRB1.

6. (canceled)

7. The method of claim 2, wherein the iPSCs are gene-edited to delete one or more HLA Class I genes, delete one or more Class II genes, and/or delete one or more genes governing HLA or MHC expression or presentation capacity.

8. The method of claim 7, wherein the iPSCs comprise a deletion of HLA-A.

9-10. (canceled)

11. The method of claim 8, wherein the iPSCs comprise a deletion of HLA-A, are homozygous for both HLA-B and HLA-C, comprise a deletion of HLA-DPB1 and HLA-DQB1, and are homozygous for HLA-DRB1.

12. The method of claim 1, wherein the HSCs and/or HSPCs are gene edited to encode a hyperresponsive EPO Receptor.

13. The method of claim 1, wherein CD34+-enrichment and endothelial-to-hematopoietic transition is induced at Day 8 to Day 15 of iPSC differentiation.

14. The method of claim 13, wherein the endothelial-to-hematopoietic transition generates an HSC population comprising one or more of long-term hematopoietic stem cells (LT-HSCs), short-term hematopoietic stem cells, and hematopoietic stem progenitor cells.

15-20. (canceled)

21. The method of claim 120, wherein the induction of endothelial-to-hematopoietic transition comprises Piezol activation: wherein the Piezol activation is by contacting the CD34+-enriched cells or fraction thereof with one or more Piezol agonists.

22-27. (canceled)

28. The method of claim 21, wherein the progenies differentiate into cells characterized by (i) CD36+/CD45+ markers and (ii) further differentiate with time into cells characterized by CD36+/CD45− markers, wherein at least 80% of the cells in (ii) are identified as CD36+.

29-38. (canceled)

39. A method for treating a patient having an inherited or acquired red cell disorder, bone marrow failure disorder, high-altitude-related physiological and pathological condition, anemia, red cell enzyme deficiencies, red cell membrane disorder, hemoglobinopathy, hemolytic anemia, nutritional anemia disorder of heme production, or hemochromatosis, the method comprising administering the erythrocyte lineage of claim 1 to the patient.

40. A hematopoietic stem cell (HSC) or erythroid progenitor composition, wherein the HSCs or erythroid progenitors express an erythropoietin receptor (EPOR) that is hyperresponsive to EPO.

41-46. (canceled)

47. The composition of claim 40, wherein the HSCs or erythroid progenitors are HLA-A^{neg}, homozygous for both HLA-B and HLA-C, and HLA-DPB1^{neg} and HLA-DQB1^{neg}.

48-56. (canceled)

57. The composition of claim 48, wherein HSCs are prepared from iPSCs in a process that comprises Piezol activation; wherein the Piezol activation is by contacting pluripotent stem cells or cells derived or differentiated therefrom with one or more Piezol agonists.

58-59. (canceled)

60. A method for making an HSC population, comprising: preparing a population of iPSCs harboring an EPOR gene encoding an EPOR that is hyperresponsive to EPO, and differentiating the iPSCs to HSCs comprising LT-HSCs.

61-74. (canceled)

75. The method of claim 60, wherein cells are subjected to Piezol activation, wherein the cells subjected to Piezol activation are selected from one or more of iPSCs, ECs, HECs, and HSCs; and wherein the Piezol activation is by contacting pluripotent stem cells or cells differentiated therefrom with one or more Piezol agonists at an effective amount.

76. The method of claim 75, wherein the effective amount of the Piezol agonist is in the range of 0.1 to 500 μM, or in the range of 0.1 to 100 μM.

77. The method of claim 76, wherein the composition comprises at least 0.0001% LT-HSCs, or at least about 10², at least about 10³, or at least about 10⁴, or at least about 10⁵, or at least about 10⁶ HSCs.

78-84. (canceled)
